(19) United States
(12) Patent Application Publication
(10) Pub. No.: US 2019/0264253 A1
(43) Pub. Date: Aug. 29, 2019

(54) ULTRASENSITIVE ELECTROCHEMICAL BIOSENSORS

(71) Applicant: MOLECULAR WAREHOUSE, LTD., Guildford (GB)

(72) Inventors: Zhong GUO, Queensland (AU); Kirill ALEXANDROV, Queensland (AU)

(21) Appl. No.: 16/342,861

(22) PCT Filed: Oct. 18, 2017
(86) PCT No.: PCT/GB2017/053154

§ 371 Date: Apr. 17, 2019

(30) Foreign Application Priority Data
Oct. 19, 2016 (GB) 1617695.0
Nov. 30, 2016 (GB) 1620322.6

(51) Int. Cl. C12Q 1/26; C12N 15/52; G01N 33/68; C12Q 1/00

(52) U.S. Cl. CPC C12Q 1/26; C12N 15/52; C12Y 101/01; C12Q 1/005; G01N 33/6845

(57) ABSTRACT

The invention relates to biosensors. More particularly, this invention relates to an electrochemical biosensor and to electrochemically active enzymes or variants thereof that are suitable for detection of one or more target molecules in a sample.

### TECHNICAL FIELD

THIS INVENTION relates to biosensors. More particularly, this invention relates to an electrochemical biosensor and to electrochemically active enzymes or variants thereof that are suitable for detection of one or more target molecules in a sample. The biosensor molecule may also relate to the field of synthetic biology such as for constructing artificial cellular or extracellular signalling networks.

### BACKGROUND

Detection of target molecules or analytes in biological samples is central to diagnostic monitoring of health and disease. Key requirements of analyte detection are specificity and sensitivity, particularly when the target molecule or analyte is in a limiting amount or concentration in a biological sample. Previous approaches include use of monoclonal antibodies which specifically bind the analyte. This type of diagnostic approach has become well known and widely used in the enzyme-linked immunosorbent sandwich assay (ELISA) format which is the gold standard for detecting specific analytes in complex biological samples.

Over the last three decades, biosensors have also become a practical alternative to complex and expensive analytical instruments used in healthcare, agriculture and environmental monitoring¹. Among several currently used detection technologies such as optical, acoustic and piezoelectric, electrochemical sensors feature prominently due to their simplicity, specificity and high performance². Electrochemical blood glucose sensors are the most commercially successful biosensors accounting for nearly 90% of the US$15 billion global biosensor market (Transparency Market Research report, titled, ‘Biosensors Market—Global Industry Analysis, Size, Share, Growth, Trends and Forecast, 2014-2020). The success of these sensors is due to their high selectivity and sensitivity combined with simplicity of design and ease of manufacturing. The sensors are based on amperometric monitoring of glucose oxidation by recombinant glucose oxidase or glucose dehydrogenase (GDH)³. The simplicity and robustness of the design enables manufacturing of disposable glucose-sensing electrodes for less than $0.1 in a continuous screen printing process⁴. The electrochemical output of the biosensor enables its connectivity with portable electronic devices such as smart phones via unsophisticated and inexpensive electronic adaptors. Remarkably, this technological and commercial success has not been paralleled by other electrochemical biosensors despite the need for better and cheaper diagnostics and analytics in many industries. This can be at least in part explained by the unique features of glucose sensing where the analyte is present at high (4-10 mM) concentration which also provides the source of energy for a selective, physically stable and highly processive electrochemical receptor.

There is a need for biosensors which are able to detect other analytes than glucose, in particular for biosensors which may be configured to detect a range of different analytes, and also for biosensors which provide increased sensitivity of detection.

### SUMMARY

The present invention addresses a need to develop quantitative, relatively inexpensive and easily produced molecular biosensors that readily detect the presence or the activity of target molecules (e.g analytes) on short time scales that are compatible with treatment regimes. Such biosensors can either be applied singly or in multiplex to validate and/or diagnose molecular phenotypes with high specificity and great statistical confidence irrespective of the genetic background and natural variations in unrelated physiological processes. Such biosensors may be used in other testing procedures such as where the target molecule or analyte is an illicit drug or performance-enhancing sub stance.

The biosensors of the present invention are further particularly suited to incorporation into electrical devices such as point-of-care devices for analysis and transmission of diagnostic results. The biosensors of the invention typically have specificity for a target molecule and produce an electrical response to detection of the target molecule. Preferred biosensors of the present invention provide high sensitivity to a target molecule through allosteric peptide-regulated reversible changes to catalytic activity which are further linked to binding of the target molecule to a binding moiety. The peptide-regulated change may be couplable to a range of different binding interactions thus allowing for detection of different target molecules based on the same common peptide-regulated architecture. The biosensors of the invention may thus be suitable for engineering for use in detection of more than one target molecule when configured with appropriate binding moieties. The biosensors of the invention typically comprise an oxidoreductase enzyme or a variant thereof.

The present invention provides an oxidoreductase enzyme comprising a heterologous amino acid sequence which is responsive to a peptide, wherein binding of the peptide to the heterologous amino acid sequence reversibly regulates catalytic activity of the enzyme. The binding of the peptide may cause a reduction in the catalytic activity of the enzyme, or may enhance the catalytic activity of the enzyme.

The invention further provides an oxidoreductase enzyme comprising a heterologous amino acid sequence inserted at a location comprising one or more residues which influence substrate binding of said enzyme, wherein the heterologous amino acid sequence reversibly regulates the catalytic activity of the enzyme.

The invention additionally provides a polypeptide comprising a first fragment sequence of an oxidoreductase enzyme, which is capable of non-covalently interacting with a polypeptide comprising a second fragment sequence of said enzyme to reconstitute a stable oxidoreductase enzyme, wherein the first and second fragment sequences represent sequences obtainable by cleavage of the enzyme at a location comprising one or more residues which influence substrate binding by said enzyme.

The invention further provides a biosensor comprising an enzyme and a heterologous amino acid sequence that releasably maintains said enzyme in a catalytically inactive state in the presence of a peptide, wherein the heterologous amino acid sequence binds to the peptide to switch the enzyme from a catalytically active state to a catalytically inactive state.

The invention also provides a biosensor comprising an oxidoreductase enzyme of the invention or the polypeptides comprising first and second fragment sequences of the invention.

The invention also provides a composition or kit comprising the oxidoreductase enzyme of the invention or a biosensor of the invention or the polypeptides comprising first and second fragment sequences of the invention. Where the composition or kit comprises said oxidoreductase enzyme or biosensor, it may further comprise a said peptide acting to regulate catalytic activity of the enzyme by binding to said heterologous amino acid sequence.

The invention additionally provides a method of detecting a target molecule, comprising contacting the oxidoreductase enzyme of the invention, a biosensor of the invention or the polypeptides comprising first and second fragment sequences of the invention with a sample under conditions suitable for detection of the presence or absence of the target molecule in the sample.

The invention further provides a method of diagnosis of a disease or condition in an organism, comprising contacting the oxidoreductase enzyme of the invention, a biosensor of the invention or the polypeptides comprising first and second fragment sequences of the invention with a sample obtained from the organism under conditions suitable for detection of the presence or absence of the target molecule in the sample, wherein presence or absence of the target molecule in the sample is indicative of whether the organism has, or is at risk of having, said disease or condition.

The invention also provides a detection device that comprises a cell or chamber that comprises the oxidoreductase enzyme of the invention, a biosensor of the invention or the polypeptides comprising first and second fragment sequences of the invention.

The invention additionally provides a nucleic acid encoding the oxidoreductase enzyme of the invention, a biosensor of the invention or a polypeptide comprising a first or second fragment sequence of the invention.

### BRIEF DESCRIPTION OF THE FIGURES

FIG. 1. Biosensor architectures based on PQQ-GDH. (A) Insertion of calmodulin (CaM) into the loop connecting A and B of the (3-sheet 3 resulted in a biosensor of Ca²⁺. (B) Separation of PQQ-GDH into two halves at the same location allows construction of two component biosensor system. Shaded half of the enzyme represents a mutant unable to support the catalysis that is displaced by the wild type version following the ligand mediated scaffolding. (C) Ribbon representation of the structure of PQQ-GDH. Ribbon representation of the enzyme in complex with PQQ and glucose. The PQQ cofactor is displayed in ball and stick representation while glucose is colored in atomic colors. The bound Ca′ is displayed as space filing object. The (3-sheets are marked with respective numbers and marked by letters. The active site residues involved in coordination of glucose are displayed in ball and stick. The arrow indicates the position of the insertion into the loop connecting β-sheets 4 and 5.

FIG. 2. Construction of CaM-BP (Calmodulin-binding peptide) inducible GDH-CaM chimera and its use for construction of a two component rapamycin biosensor. (A) schematic representation of a GDH-CaM chimera and its interaction with CaM-BP. (B) GDH activity of 10 nM GDH-CaM chimera in response to Ca²⁺ and CaM-BP. The inset shows the fit of the titration data. (C) Schematic of GDH-CaM chimera-based rapamycin receptor. (D) Titration of 10 nM of GDH-CaM-FKBP12 and 30 nM FRB-Cam-BP with increasing concentrations of rapamycin.

FIG. 3. Testing the generic nature of the two component biosensor architecture based on a CaM-GDH chimera. (A) Schematic representation of the FK506 (tacrolimus) biosensor. (B) Time resolved changes in GDH activity of 10 nM solution of GDH-CaM-FKBP12 fusion with 30 nM solution of CaM-BP calcinurin B fusion in complex with calcinurin A in the absence of presence of indicated concentrations of FK506. (C) Schematic representation of the a-amylase two component biosensor. (D) As in B but using 20 nM GDH-CaM-VHH fusion and 50 nM VHH-CaM-BP

FIG. 4. Single component sterically inhibited biosensor. (A) Schematic representation of a ligand-activated sterically auto-inhibited CaM-GDH biosensor that can be activated by either proteolysis or ligand binding (B) activity of the 10 nM solution of the biosensor shown in A upon exposure to 10 μM of PDZ peptide or of thrombin. (C) schematic representation of an improved biosensor design with PDZ binding sequences flanking the CaM-BP (D) An ultrasensitive two component biosensor architecture based on the developed autoinhibited unit.

FIG. 5. Development and applications of an OFF switch biosensor based on GDH. (A) Schematic representation of an affinity clamp operated GDH biosensor, L-denotes the ligand peptide, (B) Enzymatic activity of 10 nM solution of the affinity clamp-GDH chimera in the presence of 1 μM of a strong or 2 μM of a weak peptide ligand. (C) as in B but using the biosensor with the optimized linkers between the affinity clamp and the GDH, titrated with the increasing concentrations of the strong ligand peptide. (D) A dissociative biosensor architecture based on the developed affinity clamp-GDH biosensor. The star denotes the ligand either genetically fused or conjugated to the regulatory domain. (E) Exemplification of the design shown in D with a biosensor for IL18: the ligand is IL18 and the binder is IL18 binding protein. (F) Enzymatic activity of 10 nM solution of the affinity clamp-GDH IL18 binding protein chimera (SEQ ID NO: 37) mixed with 50 nM solution of the fusion of IL18 with the affinity-clamp ligand (SEQ ID NO: 38), at varying concentrations of IL18 (top to bottom traces from 0 to 2.5 μM IL18). (G) As in D but using a binder that competitively associates with the receptor domain and is dislodged by the binding of the ligand.

FIG. 6. (A) Protease activatable autoinhibited biosensor module based on the developed affinity clamp-operated GDH. (B) Activity of autoinhibited module form A carrying TVMV cleavage site between the enzyme and Affinity clamp binding peptide (strong ePDZ ligand): Seq ID NO 39) in the absence or presence of TVMV protease. In the experiment 10 nM of the fusion protein of Seq ID NO 39 was preincubated with 2 μM of TVMV protease for 2 hours and analysed for activity in the buffer containing 50 μM CaCl₂), 0.6 mM PMS, 0.06 mM DCPIP, 20 mM Glucose. (C) As in B but using the fusion protein with a weak binding ePDZ peptide (SEQ ID NO 40). A further control reaction was supplemented with 2 μM of the strong binding ePDZ peptide. (D) 10 nM of fusion protein with the weak binding ePDZ peptide (SEQ ID NO: 40) in the presence of the indicated concentrations of TVMV protease. (E)Schematic representation of ultrasensitive two component biosensor based on the module pictured in A.

FIG. 7. Comparison of the activation rates of the split and GDH-CaM-based versions of rapamycin biosensors. Split version: A solution of 10 nM of a fusion of mutational inactivated N-terminal GDH fragment fused to TVMV cleavage site-FKBP- and wild type C-terminal portion of GDH, as described in Examples 1 and 5 (SEQ ID NO: 44). The protein was pre-cleaved with TVMV prior to the assay. The former solution was mixed with 15 nM solution of N-terminal GDH-FRB, SEQ ID NO: 43. The GDH-CaM rapamycin biosensor was: 10 nM GDH-CalM-FKBP (SEQ ID NO: 11), 2.5 μM FRB-CalM BP (SEQ ID NO:12).

All assays contained: 50 μM CaCl₂), 0.6 mM PMS, 0.06 mM DCPIP, 20 mM Glucose. The graph shows the activation rate and amplitude of the two GDH based biosensors. The data represents kobs (min⁻¹) of the individual reaction plotted against the concentration of rapamycin (in μM).

### DESCRIPTION OF THE SEQUENCES

SEQ ID NO: 1 is the amino acid sequence of a mature PQQ-GDH polypeptide.

SEQ ID NO: 2 is the full length amino acid sequence of a PQQ_GDH polypeptide.

SEQ ID NO: 3 is the amino acid sequence of a calmodulin protein.

SEQ ID NO: 4 is the amino acid sequence of a calmodulin binding peptide, which binds SEQ ID NO: 3.

SEQ ID NO: 5 is the amino acid sequence of a modified calmodulin binding peptide.

SEQ ID NO: 6 is the amino acid sequence of a modified calmodulin binding peptide.

SEQ ID NO: 7 is the amino acid sequence of a modified calmodulin binding peptide.

SEQ ID NO: 8 is the amino acid sequence of a modified calmodulin binding peptide.

SEQ ID NO: 9 is the amino acid sequence of a GDH-calmodulin fusion protein (first generation).

SEQ ID NO: 10 is the amino acid sequence of a GDH-calmodulin fusion protein (second generation).

SEQ ID NO: 11 is the amino acid sequence of a GDH-calmodulin-FKP12 fusion protein.

SEQ ID NO: 12 is the amino acid sequence of a calmodulin binding peptide-FRB fusion protein.

SEQ ID NO: 13 is the amino acid sequence of an FKBP12 protein.

SEQ ID NO: 14 is the amino acid sequence of an FRB protein.

SEQ ID NO: 15 is the amino acid sequence of a SUMO-calcineurin alpha fusion protein.

SEQ ID NO: 16 is the amino acid sequence of a calcineurin beta-calmodulin binding peptide fusion protein.

SEQ ID NO: 17 is the amino acid sequence of a SUMO tag.

SEQ ID NO: 18 is the amino acid sequence of a calcineurin alpha protein

SEQ ID NO: 19 is the amino acid sequence of a calcineurin beta protein SEQ ID NO: 20 is the amino acid sequence of a GDH-calmodulin-VHH1 fusion protein.

SEQ ID NO: 21 is the amino acid sequence of a VHH2-calmodulin binding peptide fusion protein.

SEQ ID NO: 22 is the amino acid sequence of a VHH1 antibody.

SEQ ID NO: 23 is the amino acid sequence of a VHH2 antibody.

SEQ ID NO: 24 is the amino acid sequence of a GDH-ePDZ fusion protein (first generation).

SEQ ID NO: 25 is the amino acid sequence of a GDH-ePDZ fusion protein (second generation).

SEQ ID NO: 26 is the amino acid sequence of an ePDZ protein.

SEQ ID NO: 27 is the amino acid sequence of a PDZ binding peptide (strong ligand).

SEQ ID NO: 28 is the amino acid sequence of a PDZ binding peptide (strong ligand).

SEQ ID NO: 29 is the amino acid sequence of a GDH-ePDZ-CaM-thrombin cleavage site-CaM-BP-PDZ binding peptide fusion protein.

SEQ ID NO: 30 is the amino acid sequence of a GDH fragment polypeptide (residues 1-330 of SEQ ID NO:2).

SEQ ID NO: 31 is the amino acid sequence of a GDH fragment polypeptide (residues 331-457 of SEQ ID NO:2).

SEQ ID NO: 32 is the amino acid sequence of a TVMV cleavage site.

SEQ ID NO: 33 is the amino acid sequence of a thrombin cleavage site.

SEQ ID NO: 34 is the amino acid sequence of a Factor Xa cleavage site.

SEQ ID NO: 35 is the amino acid sequence of a Factor Xa cleavage site.

SEQ ID NO: 36 is the amino acid sequence of a thrombin high affinity binding site.

SEQ ID NO: 37 is the amino acid sequence of a GDH-ePDZ-IL18 binding protein fusion protein.

SEQ ID NO: 38 is the amino acid sequence of an IL-18-ePDZ peptide fusion.

SEQ ID NO: 39 is the amino acid sequence of an autoinhibited GDH-ePDZ-strong ePDZ peptide fusion protein.

SEQ ID NO: 40 is the amino acid sequence of an autoinhibited GDH-ePDZ-weak ePDZ peptide fusion protein.

SEQ ID NO: 41 is the amino acid sequence of an IL-18 binding protein.

SEQ ID NO: 42 is the amino acid sequence of an IL-18 protein.

SEQ ID NO: 43 is the amino acid sequence of a GDH fragment polypeptide (residues 1-153 of SEQ ID NO:2) with a C-terminal FRB fusion.

SEQ ID NO: 44 is the amino acid sequence of a GDH fusion polypeptide with a catalytically inactivated N-terminal GDH fragment fused to TVMV cleavage site-FKBP- and wild type C-terminal portion of GDH.

### DETAILED DESCRIPTION

Oxidoreductase Enzyme and Catalytic Activity

The oxidoreductase enzyme of the invention comprises a heterologous amino acid sequence which is responsive to a peptide, wherein binding of the peptide to the heterologous amino acid sequence reversibly regulates catalytic activity of the enzyme. The oxidoreductase enzyme of the invention is thus engineered to be switchable from a state of reduced catalytic activity to a more catalytically active state in response based on whether the peptide is bound. The oxidoreductase enzyme is typically further engineered such that catalytic activity of the enzyme is further regulated by binding of a target molecule other than the peptide, where the target molecule is typically an analyte to be detected. The binding of both the peptide and the target molecule may be necessary for regulation of catalytic activity. In this way, the oxidoreductase enzyme may be able to be configured for detection of more than one different analyte, such as two or more, three or more, five or more or ten or more different analytes. The oxidoreductase is configured to detect each different analyte by incorporation of a suitable binding moiety able to interact with a respective binding moiety in the presence of the relevant analyte. Alternative binding moieties are then engineered into the oxidoreductase for detection of another analyte of interest. Typically, the oxidoreductase enzyme is suitable for detection of one or more analytes other than calcium ions.

The heterologous amino acid sequence releasably maintains the enzyme in a state of reduced catalytic activity. It may be responsive to binding of the peptide to switch the enzyme from a state of reduced catalytic activity to a more catalytically active state. Alternatively, binding of the peptide to the heterologous amino acid sequence releasably maintains the enzyme in a state of reduced catalytic activity. Loss of binding of the peptide may then switch the enzyme from a state of reduced catalytic activity to a more catalytically active state. Thus, binding of the peptide to the heterologous amino acid sequence reversibly regulates catalytic activity of the enzyme. The heterologous amino acid sequence may be displaced in the presence or absence of the peptide and optionally also in the further presence of a target molecule, to thereby catalytically activate the enzyme. The heterologous amino acid sequence can thus allosterically regulate the catalytic activity of the enzyme.

An oxidoreductase “enzyme” is a protein capable of displaying catalytic activity towards a substrate molecule to thereby produce one or more electrons. The enzyme may be any enzyme capable of reacting with a substrate molecule to thereby produce one or more electrons. Preferably, the enzyme is an oxidoreductase such as a GDH, glucose oxidase, LDH or DHFR. In some embodiments, the enzyme is an oxidoreductase and the activity is oxidoreductase activity. Preferably the enzyme is glucose dehydrogenase (GDH) and the substrate molecule is glucose. The catalytic activity may thus be glucose dehydrogenase activity which may be measured in accordance with Example 1. The glucose dehydrogenase may be a PQQ-GDH or an FAD-GDH. Preferably, the GDH is a PQQ-GDH. A PQQ-GDH preferably comprises the sequence of SEQ ID NO: 1 or a variant thereof. A PQQ-GDH may be encoded by a nucleic acid sequence encoding SEQ ID NO: 1 or 2.

In another embodiment the enzyme is glucose oxidase and the substrate is glucose. In another embodiment the enzyme is dihydrofolate reductase (DHFR) and the substrate molecule is dihydrofolic acid. In another embodiment the enzyme is lactate dehydrogenase (LDH) and the substrate molecule is lactate.

The oxidoreductase enzyme has a reduced or enhanced state of catalytic activity when the peptide is bound to the heterologous amino acid sequence. In some embodiments the oxidoreductase enzyme has a reduced or enhanced state of catalytic activity when the peptide is bound to the heterologous amino acid sequence, and a target molecule is also bound to a binding moiety. The reduction or enhancement of catalytic activity may be of any magnitude. The reduction or enhancement of catalytic activity is typically of a magnitude sufficient to allow for correlation with the presence of the peptide or the presence of both the peptide and target molecule. The skilled person is able to determine whether binding of the peptide regulates catalytic activity of the enzyme by comparing the activity of the enzyme with and without the peptide.

The enzyme may be described as being “catalytically active” or in a “catalytically active state” in the presence of the peptide or in the presence of the peptide and the target molecule. Alternatively, the enzyme may be described as being “catalytically inactive” or in a “catalytically inactive state” in the presence of the peptide or in the presence of the peptide and the target molecule. It should be understood that wild-type catalytic activity may not be conferred by binding or displacement of the peptide or by binding or displacement of the peptide and binding of the target molecule. Likewise, catalytic activity may not be abolished completely by binding or displacement of the peptide or by binding or displacement of the peptide and binding of the target molecule. Typically, an enzyme is catalytically active or in a catalytically active state if it is capable of displaying specific enzyme activity towards a substrate molecule to produce one or more electrons under appropriate reaction conditions. As generally used herein “catalytically inactive” and “catalytically inactive state” may refer to an enzyme that is substantially incapable of displaying specific enzyme activity towards a substrate molecule under appropriate reaction conditions. Typically, the electrons produced would be substantially less compared to that produced by a corresponding catalytically active enzyme. Electron production may be entirely absent.

The oxidoreductase enzymes and biosensors described herein produce electrons by reacting with substrate molecules in response to binding, interacting with or otherwise detecting one or more target molecules. In this context “react”, “reaction” or “reacting” with a substrate molecule means enzymatically transforming the substrate molecule into one or more product molecules with a net or overall production of one or a plurality of electrons per substrate molecule. Accordingly, the biosensor acts as an electron donor, whereby the electrons produced by the reaction may flow either directly or via an electron shuttle such as, but not limited to, phenazine methosulfate or potassium ferrocyanide, to thereby act as an anode. The resulting change in potential between anode and cathode may be detected by an electronic detector.

In some embodiments the oxidoreductase enzymes and biosensors described herein may be attached to an electrode. The mode of attachment may permit direct electron transfer from the oxidoreductase enzyme or biosensor to the electrode. Typically, the biosensor or enzyme acts as an electron donor and electrons produced by the reaction may flow directly to the electrode to form the anode. The electrode may be composed of carbon nanotubes or graphene. The oxidoreductase enzyme or biosensor may be attached to the electrode surface using 1-pyrenebutanoic acid succinimidyl ester (PBSE) as a hetero-bifunctional linker, wherein the active ester groups of the PB SE linker may react with the amino groups of lysine residues in the oxidoreductase enzyme or biosensor.

In some embodiments the oxidoreductase enzymes and biosensors described herein may be integrated into semiconductor electronic devices. Typically, the biosensor or enzyme acts as an electron donor and electrons produced by the reaction may flow either directly or via an electron shuttle such as, but not limited to, phenazine methosulfate or potassium ferrocyanide, to the semiconductor. The enzyme or biosensor may be integrated into an electrolyte-insulator-semiconductor (EIS) chip. For example, the enzyme or biosensor may be attached to a Ta₂O₅ surface. Ta₂O₅ is a pH-sensitive material and allows pH changes resulting from reaction of the biosensor or enzyme with a substrate to produce an acidic or basic product to be detected.

Heterologous Amino Acid Sequence

The heterologous amino acid sequence is typically provided as an insert within the amino acid sequence of the oxidoreductase enzyme. However fusions of the heterologous amino acid sequence at the N- or C-terminus are also possible.

Where provided as an insert, the heterologous amino acid sequence is therefore typically and contiguous with, respective portions, sub-sequences or fragments of said enzyme. The insertion is made at a position in the amino acid sequence of the enzyme which tolerates said insertion without steric clashes preventing stable folding of the enzyme. Linker sequences may be added between the insert and the sequence of the enzyme to assist toleration of the insertion. The insertion may be located at a loop or turn region in the structure of the enzyme which functionally tolerates the heterologous, sensor amino acid sequence. The insertion may be located in a region of the enzyme (such as a loop or turn region) which comprises one or more amino acid residues which influence substrate binding and/or catalytic activity of the enzyme. The insertion may thus displace one or more residues which influence substrate binding and/or catalytic activity of the enzyme, such that catalytic activity of the enzyme is regulated by the heterologous amino acid sequence. The insertion may displace one more residues which influence substrate binding and one or more residues which influence catalytic activity. The insertion may prevent or reduce substrate binding to the enzyme and/or may switch the enzyme to a state of reduced catalytic activity or a catalytically inactive state. As discussed above, the binding of the peptide to the heterologous amino acid sequence or of the peptide to the heterologous amino acid sequence and the target molecule to its binding moiety reversibly regulates catalytic activity of the enzyme. The binding of the peptide or of the peptide and the target molecule may thus reverse the displacement of one or more residues which influence substrate binding and/or catalytic activity of the enzyme by the heterologous amino acid sequence, or cause said displacement.

The catalytic activity of the enzyme may accordingly be regulated by the conformational status of the heterologous amino acid sequence, as affected by binding of the peptide or binding of the peptide and binding of the target molecule to its binding moiety. The insertion thus typically allows for the heterologous amino acid sequence to reversibly regulate catalytic activity through inducing a conformational change in the enzyme, typically at the substrate binding region and/or active site of the enzyme. The heterologous amino acid sequence typically undergoes a conformational change in the presence of the peptide or in the presence of the peptide and the target molecule which acts to regulate catalytic activity of the enzyme. The heterologous amino acid sequence may thus allosterically regulate the catalytic activity of the enzyme in the presence of the peptide or in the presence of the peptide and the target molecule.

In a preferred embodiment, the heterologous amino acid sequence is inserted in a GDH enzyme at a loop or turn region corresponding to the loop connecting beta-sheets 4 and 5 of PQQ-GDH of SEQ ID NO:1, or in a region corresponding to residues 326 to 335 of said enzyme. Preferably said insertion is made in a region corresponding to residues 328 to 332. Most preferably, the insertion is made at position 330 of SEQ ID NO:1 or at a corresponding position in another enzyme. The above insertion may delete the amino acid at position 330 of SEQ ID NO: 1 or at a corresponding position thereto. The skilled person is able to identify corresponding locations in other enzymes from structural analysis and sequence alignment. A corresponding location is typically one which accommodates the inserted heterologous amino acid sequence such that it reversibly regulates catalytic activity of the enzyme as described above. The insertion may dislocate one or more residues which form part of the glucose binding site of a GDH, such as residues corresponding to Trp346 and/or Tyr348 of SEQ ID NO:1. The insertion may additionally or alternatively dislocate a residue which forms part of a cofactor binding site of a GDH, such as a PQQ binding site, such as a residue corresponding to Thr348 of SEQ ID NO:1. The invention thus provides a GDH enzyme comprising a heterologous amino acid sequence inserted within residues 326-335 of, such as residues 328-332 of, including at position 330 of, SEQ ID NO:1 or a variant thereof. Such an enzyme may comprise in order the sequences of SEQ ID NO: 30 (residues 1-329) or a variant thereof, the heterologous amino acid sequence, and SEQ ID NO: 31 (residues 331-454) or a variant thereof. Variants of SEQ ID Nos 1, 30 and 31 are further described below. The above sequences may be separated by linker sequences allowing for toleration of the inserted heterologous amino acid sequence as described above. In some embodiments, the heterologous amino acid sequence is not inserted in a GDH enzyme at a loop or turn region corresponding to the loop connecting beta-sheets 3A and 3B of a PQQ-GDH of SEQ ID NO: 1 or a region corresponding to residues 153-155 of said enzyme, or a corresponding region thereto.

The heterologous amino acid sequence may be any binding moiety for any peptide. Exemplary heterologous amino acid sequences and peptides are described below. The heterologous amino acid sequence is preferably an amino acid sequence of a calcium-binding protein, or a functional fragment thereof. The calcium binding protein may be a calmodulin or a functional calcium-binding fragment thereof. The calcium-binding protein may be calmodulin of SEQ ID NO 3 or a variant thereof. Where the heterologous amino acid sequence is calmodulin of SEQ ID NO 3 or a variant thereof, the peptide binding thereto is typically a calmodulin-binding peptide. The calmodulin-binding peptide may comprise SEQ ID NO: 4 or a variant thereof. Variants thereof suitably retain calmodulin-binding activity, and may include any of SEQ ID NOs 5-8. Preferably, the calmodulin-binding peptide comprises or consists of SEQ ID NO 8, which has reduced calmodulin-binding activity, or a variant thereof. The invention also provides the above calmodulin-binding peptides and variants as peptides per se.

SEQ ID NO 8 or a variant thereof retaining reduced calmodulin-binding is preferred for providing lower affinity interaction which may then be cooperatively enhanced by further binding of a target molecule to a binding moiety. By “cooperative enhancement” is meant that catalytic activity is measurably enhanced or reduced only in the presence of both the peptide and the target molecule. The affinity of the heterologous amino acid sequence for the peptide and/or of the target molecule for its binding moiety may be at least an order of magnitude higher in the presence of both the peptide and the target molecule as compared to only the peptide or the target molecule.

Where the heterologous amino acid sequence is a calcium-binding protein or functional fragment thereof, regulation of catalytic activity of the enzyme typically requires the presence of calcium ions in addition to the peptide, or in addition to the peptide and the target molecule. Such an oxidoreductase enzyme typically does not display regulation of catalytic activity by the heterologous amino acid sequence in the presence of calcium alone, absent the peptide or the peptide and the target molecule. The invention preferably provides an oxidoreductase enzyme based on PQQ-GDH and calmodulin as described below comprising SEQ ID NO: 9 or 10 or a variant thereof. An oxidoreductase enzyme comprising SEQ ID NO: 10 is particularly preferred.

Another preferred heterologous amino acid sequence is an affinity clamp. The affinity clamp may be the ePDZ domain of SEQ ID NO 26 or a variant thereof. In this embodiment, the peptide is preferably the PDZ domain-binding peptide of SEQ ID NO 27 or 28 or a variant of either thereof. The invention further provides an oxidoreductase enzyme based on PQQ-GDH and ePDZ as described below comprising SEQ ID NO: 24 or 25 or a variant thereof. An oxidoreductase enzyme comprising SEQ ID NO: 25 is particularly preferred.

The peptide may be any peptide binding moiety described herein which is able to bind to the heterologous amino acid sequence, including suitable peptides listed as “binding moieties” below. Preferred peptides include calmodulin-binding peptides and peptides binding affinity clamps as described herein and in the Examples. Other possible peptides include SH3:SH3 domain binding peptide, antibody: antibody binding peptide, two leucine zipper peptides. The peptide may comprise a further binding moiety for a binding partner (respective binding moiety) other than the heterologous amino acid sequence, as discussed below. The peptide binding to the heterologous amino acid sequence may be provided as a separate molecule to the enzyme (and thus as a further component of a biosensor), or alternatively may form part of the contiguous amino acid sequence of said enzyme. In the latter embodiment, the peptide is located at a position from which it is able to interact with the heterologous amino acid sequence under conditions promoting such interaction. The peptide may be comprised as an insert within the amino acid sequence of the enzyme or as a C-terminal or N-terminal fusion thereto.

The oxidoreductase enzyme may further comprise respective binding moieties whose binding prevents interaction between the peptide and the heterologous amino acid sequence. Any pair of respective binding moieties may be used. This allows for autoinhibition of enzyme activity. Disruption of the interaction between the respective binding moieties can then provide for regulation of catalytic activity by the peptide. A ligand competing for binding with one of the respective binding moieties may be provided such that their interaction is disrupted. Multiple binding moieties may be provided such that disruption of more than one interaction is required for regulation of catalytic activity by the peptide, also reducing spontaneous activation. A protease cleavage site may be provided, suitably adjacent to one of the binding moieties, such that provision of a cognate protease allows for cleavage of the site to release the enzyme from autoinhibition by the interaction between the binding moieties. The peptide is thereby released to interact with the heterologous amino acid sequence.

Where the peptide is a calmodulin-binding peptide and the heterologous amino acid sequence is a calmodulin protein located in a GDH enzyme at a loop or turn region corresponding to the loop connecting beta-sheets 4 and 5 of PQQ-GDH of SEQ ID NO:1 (or a related location discussed above), the peptide may be provided C-terminally in the enzyme, preferably C-terminal to the native C-terminal GDH enzyme sequence. The invention provides in this embodiment a GDH enzyme comprising in order the sequences of SEQ ID NO: 30 (residues 1-329) or a variant thereof, the sequence of SEQ ID NO: 3 or a variant thereof, the sequence of SEQ ID NO: 31 (residues 331-454) or a variant thereof, and the sequence of SEQ ID NO 4 or a variant thereof, preferably SEQ ID NO: 8 or a variant thereof. The above sequences may be separated by linker sequences allowing for toleration of the inserted heterologous amino acid sequence as described above. The above series of sequences may be flanked N- and C-terminally by respective binding moieties preventing interaction between the peptide and the calmodulin protein. The N-terminal binding moiety may be the ePDZ domain of SEQ ID NO 26 and the C-terminal binding moiety the PDZ domain binding peptide of SEQ ID NO 27 or 28 or a variant of either thereof. In this embodiment, the invention further provides the oxidoreductase enzyme described below comprising SEQ ID NO: 29 or a variant thereof.

Target Molecule and Binding Moieties

Whilst the catalytic activity of the oxidoreductase enzyme of the invention may be solely regulated by binding of the peptide to the heterologous amino acid sequence, preferably it is further regulated by interaction of one or more binding moieties, typically further dependent on presence of a target molecule. In this manner, presence of a target molecule other than the peptide may be detected, with enhancement or reduction of catalytic activity then being indicative of the presence of the target molecule. Binding of the target molecule may cooperatively enhance regulation of the catalytic activity of the enzyme as described above.

The oxidoreductase enzyme may comprise a binding moiety and the peptide a respective binding moiety therefor such that interaction between the binding moieties regulates binding of the peptide to the heterologous amino acid sequence. The interaction between the binding moieties may induce a conformational change in the enzyme. The interaction between the binding moieties may further depend on the presence of a target molecule. The respective binding moieties may thus brought into association by the target molecule in a binding complex. The target molecule may be any target molecule described herein, and the binding moiety(ies) any that provide for binding thereof. The binding moiety is typically comprised in the oxidoreductase enzyme such that binding of the target molecule can effect a conformational change in the enzyme,

As generally used herein a “binding moiety” or “binding moieties” refer to one or a plurality of molecules or biological or chemical components or entities that are capable of recognizing and/or binding each other, and/or one or more other target molecules. Binding moieties may be proteins, nucleic acids (e.g single-stranded or double-stranded DNA or RNA), sugars, oligosaccharides, polysaccharides or other carbohydrates, lipids or any combinations of these such as glycoproteins, PNA constructs etc or molecular components thereof. By way of example only, binding moieties may be, or comprise: (i) an amino acid sequence of a ligand binding domain of a receptor responsive to binding of a target molecule such as a cognate growth factor, cytokine, a hormone (e.g. insulin), neurotransmitters etc; (ii) an amino acid sequence of an ion or metabolite transporter capable of, or responsive to, binding of a target molecule such as an ion or metabolite (e.g a Ca²⁺-binding protein such as calmodulin or calcineurin or a glucose transporter); (iii) a zinc finger amino acid sequence responsive to zinc-dependent binding a DNA target molecule; (iv) a helix-loop-helix amino acid sequence responsive to binding a DNA target molecule; (v) a pleckstrin homology domain amino acid sequence responsive to binding of a phosphoinositide target molecule; (vi) an amino acid sequence of a Src homology 2- or Src homology 3-domain responsive to a signaling protein; (vii) an amino acid sequence of an antigen responsive to binding of an antibody target molecule; or (viii) an amino acid sequence of a protein kinase or phosphatase responsive to binding of a phosphorylatable or phosphorylated target molecule; (ix) ubiquitin-binding domains; (x) proteins or protein domains that bind small molecules, drugs or antibiotics such as rapamycin-binding FKBP and FRB domains; (xi) single- or double-stranded DNA, RNA or PNA constructs that bind nucleic acid target molecules, such as where the DNA or RNA are coupled or cross-linked to an amino acid sequence or other protein-nucleic acid interaction; and/or (xii) an affinity clamp such as a PDZ-FH3 domain fusion; inclusive of modified or engineered versions thereof, although without limitation thereto.

Particular binding moieties of use in the invention are provided by SEQ ID NOs 3-8, 13-14, 15-16, 18-19, 22-23 and 26-28 and variants thereof. Variants are typically functionally binding variants for the relevant respective binding moiety. Combinations of such binding moieties forming respective binding moieties are provided in the Examples and described further herein.

It will also be appreciated that binding moieties may be modified or chemically derivatized such as with binding agents such as biotin, avidin, epitope tags, lectins, carbohydrates, lipids although without limitation thereto.

In one embodiment, the binding moieties comprise an amino acid sequence of at least a fragment of any protein or protein fragment or domain that can bind or interact directly, or bind to a target molecule. The binding moiety may be, or comprise a protein such as a peptide, antibody, antibody fragment or any other protein scaffold that can be suitably engineered to create or comprise a binding portion, domain or region (e.g. reviewed in Binz et al., 2005 Nature Biotechnology, 23, 1257-68.) which binds a target molecule.

In one particular embodiment, the binding moieties respectively are, or comprise, amino acid sequences of an affinity clamp. The affinity clamp preferably comprises a recognition domain and, optionally, an enhancer domain. The recognition domain is typically capable of binding one or more target molecules, such as described in (i)-(ix) above. Recognition domains may include, but are not limited to, domains involved in phospho-tyrosine binding (e.g. SH2, PTB), phospho-serine binding (e.g. UIM, GAT, CUE, BTB/POZ, VHS, UBA, RING, HECT, WW, 14-3-3, Polo-box), phospho-threonine binding (e.g. FHA, WW, Polo-box), proline-rich region binding (e.g. EVH1, SH3, GYF), acetylated lysine binding (e.g. Bromo), methylated lysine binding (e.g. Chromo, PHD), apoptosis (e.g. BIR, TRAF, DED, Death, CARD, BH), cytoskeleton modulation (e.g. ADF, GEL, DH, CH, FH2), ubiquitin-binding domains or modified or engineered versions thereof, or other cellular functions (e.g. EH, CC, VHL, TUDOR, PUF Repeat, PAS, MH1, LRR1, IQ, HEAT, GRIP, TUBBY, SNARE, TPR, TIR, START, SOCS Box, SAM, RGS, PDZ, PB1, LIM, F-BOX, ENTH, EF-Hand, SHADOW, ARM, ANK).

The enhancer domain typically increases or enhances the binding affinity for at least one or the target molecules. In some embodiments, the affinity may be increased by at least 10, 100 or 1000 fold compared to that of the recognition domain alone. The affinity clamp may further comprise linker connecting the recognition domain and the enhancer domain.

In one particular embodiment, the affinity clamp comprises a recognition domain that comprises at least a portion or fragment of a PDZ domain and an enhancer domain that comprises at least a portion or fragment of a fibronectin type III domain. The PDZ domain may be derived from a human Erbin protein. Erbin-PDZ (ePDZ) binds to target molecules such as the C-termini of p120-related catenins (such as δ-catenin and Armadillo repeat gene deleted in Velo-cardio-facial syndrome (ARVCF)). Preferably, this embodiment of the affinity clamp further comprises the tenth (10^{th}) type III (FN3) domain of human fibronectin as an enhancer domain.

In some embodiments, the affinity clamp may comprise one or more connector amino acid sequences. For example, a connector amino acid sequence may connect the protease amino acid sequence (such as comprising a protease amino acid sequence) to the Erbin-PDZ domain, the Erbin-PDZ domain to the FN3 domain and/or the FN3 domain to the inhibitor.

Reference is also made to WO2009/062170, Zhuang & Liu, 2011, Comput. Theoret. Chem. 963 448, Huang et al, 2009, J. Mol. Biol. 392 1221, Huang et al., 2008, PNAS (USA) 105 6578, and Koidel,* and Huang Methods Enzymol. 2013; 523: 285-302 for a more detailed explanation of affinity clamp structure and function, and of particular affinity clamps that may be used in accordance with the invention. An example of an affinity clamp that may be employed in the invention and target peptides therefor are provided as SEQ ID NOs: 26 and SEQ ID NOs 27 and 28.

The above discussion of affinity clamps and target molecules therefor also applies to selection of an affinity clamp as a heterologous amino acid sequence and selection of a binding peptide therefor.

In another embodiment, the binding moieties comprise one or a plurality of epitopes that can be bind or be bound by an antibody target molecule.

In another embodiment, the binding moieties may be or comprise an antibody or antibody fragment, inclusive of monoclonal and polyclonal antibodies, recombinant antibodies, Fab and Fab′2 fragments, diabodies and single chain antibody fragments (e.g. scVs), although without limitation thereto. Suitably, the first and second binding moieties may be or comprise respective antibodies or antibody fragments that bind a target molecule.

In yet another particular embodiment, the binding moieties may be or comprise an antibody-binding molecule, wherein the antibody(ies) has specificity for a target molecule. The antibody-binding molecule is preferably an amino acid sequence of protein A, or a fragment thereof (e.g a ZZ domain), which binds an Fc portion of the antibody.

The target molecule may be any ligand, analyte, small organic molecule, epitope, domain, fragment, subunit, moiety or combination thereof, such as a protein inclusive of antibodies and antibody fragments, antigens, enzymes, phosphoproteins, glycoproteins, lipoproteins and glycoproteins, lipid, phospholipids, carbohydrates inclusive of simple sugars, disaccharides and polysaccharides, nucleic acids, nucleoprotein or any other molecule or analyte. These include drugs and other pharmaceuticals including antibiotics, banned substances, illicit drugs or drugs of addiction, chemotherapeutic agents and lead compounds in drug design and screening, molecules and analytes typically found in biological samples such as biomarkers, tumour and other antigens, receptors, DNA-binding proteins inclusive of transcription factors, hormones, neurotransmitters, growth factors, cytokines, receptors, metabolic enzymes, signaling molecules, nucleic acids such as DNA and RNA, membrane lipids and other cellular components, pathogen-derived molecules inclusive of viral, bacterial, protozoan, fungal and worm proteins, lipids, carbohydrates and nucleic acids, although without limitation thereto. As previously, described, it will be appreciated that the “same” target molecule can be bound by different, respective binding moieties.

In some embodiments, the target molecule is an enzyme such as a amylase. In such embodiments, the first and second binding moieties may be antibodies therefor, such as exemplified camelid antibodies VHH1 and VHH2 comprising the sequences of SEQ ID NOs: 22 and 23 or variants thereof. Such variants suitably retain a amylase-binding activity.

In some embodiments, the target molecule is a small organic molecule such as rapamycin. In such embodiments, the first and second binding moieties may be, respectively an FKBP and FRB. A preferred FKBP and FRB pair comprises the sequences of SEQ ID NOs: 13 and 14 or variants thereof.

In some embodiments, the target molecule is a small organic molecule such as FK506. In such embodiments, the first and second binding moieties may be, respectively, an FKBP and a Calcineurin AB complex. Examples of these binding moieties are provided as SEQ ID NOs 13, 18 and 19 or a variant thereof. SEQ ID NO: 19 or a variant thereof may be fused with a calmodulin-binding peptide described herein such as SEQ ID NO: 8 or a variant thereof. Such a fusion protein may comprise SEQ ID NO: 16 or a variant thereof.

In some embodiments, the target molecule is a cytokine, such as IL-18. In such embodiments, the first and second binding moieties may be, respectively the cytokine and a cytokine-binding protein. Examples of such binding moieties are provided by the cytokine IL-18 (SEQ ID NO: 42) and IL-18 binding protein (SEQ ID NO: 41). An example of use of these binding moieties is provided by the dissociative sensor of SEQ ID NO: 37, incorporating an ePDZ domain and an IL-18 binding protein, and the fusion peptide of SEQ ID NO: 38, incorporating an ePDZ peptide and IL-18.

Particular oxidoreductase and peptide fusion molecules incorporating respective binding moieties as described above are provided as SEQ ID Nos 11-12, 16, 20-21, 24-25 and 29, and SEQ ID NOs 37-38 or variants thereof.

### Autoinhibited Enzymes/Protease Activation

The above-described oxidoreductase (such as GDH) enzymes may comprise one or more protease cleavage sites, wherein cleavage of a said site by a protease displaces the inhibitory moiety to activate catalytic activity of the enzyme. The enzyme may further comprise a sequence enhancing binding and/or cleavage efficiency of the protease. An example of such a sequence enhancing thrombin binding is provided as SEQ ID NO: 36 or a variant thereof.

The oxidoreductase enzyme may comprise a binding moiety capable of interacting with a respective binding moiety on a further molecule, wherein interaction between the binding moieties displaces the inhibitory moiety to activate catalytic activity of the enzyme. Such an oxidoreductase enzyme may further comprise one or more protease cleavage sites, wherein the further molecule additionally comprises a protease and interaction between the binding moieties acts to bring the protease into proximity with a said site to cleave said site and displace the inhibitory moiety. The binding moieties and protease cleavage site(s) may be selected from any of those described herein. In an embodiment where the oxidoreductase enzyme displays a reduction in catalytic activity in the presence of binding of said peptide to the heterologous amino acid sequence, as described above, the oxidoreductase enzyme may comprise one or protease cleavage sites, where cleavage of a said site by a protease releases said peptide to thereby enhance catalytic activity of the enzyme. The protease may comprise a binding moiety for a respective binding moiety in said enzyme so as to bring it into proximity to the enzyme for cleavage of said site. The interaction between the binding moieties may be dependent on the presence of a target molecule.

A “protease” is a protein which displays, or is capable of displaying, an ability to hydrolyse or otherwise cleave a peptide bond. Like terms include “proteinase” and “peptidase”. Proteases include serine proteases, cysteine proteases, metalloproteases, threonine proteases, aspartate proteases, glutamic acid proteases, acid proteases, neutral proteases, alkaline proteases, exoproteases, aminopeptidases and endopeptidases although without limitation thereto. Proteases may be purified or synthetic (e.g. recombinant synthetic) forms of naturally-occurring proteases or may be engineered or modified proteases which comprise one or more fragments or domains of naturally-occurring proteases which, optionally, have been further modified to possess one or more desired characteristics, activities or properties.

The target protease may be any protease for which a protease cleavage site is known. Suitably, the target protease is detectable in a biological sample obtainable from an organism, inclusive of bacteria, plants and animals. Animals may include humans and other mammals. Non-limiting examples of target proteases include proteases involved in blood coagulation such as thrombin, plasmin, factor VII, factor IX, factor X, factor Xa, factor XI, factor XII (Hageman factor) and other proteases such as kallikreins (e.g. kallikrein III, P-30 or prostate specific antigen), matrix metalloproteinases (such as involved in wounds and ulcers; e.g. MMP7 and MMP9), adamalysins, serralysins, astacins and other proteases of the metzincin superfamily, trypsin, chymotrypsin, elastase, cathepsin G, pepsin and carboxypeptidase A as well as proteases of pathogenic viruses such as HIV protease, West Nile NS3 protease and dengue virus protease although without limitation thereto.

The protease amino acid sequence may be an entire amino acid sequence of a protease or may be an amino acid sequence of a proteolytically-active fragment or sub-sequence of a protease. In some embodiments, the protease may be an autoinhibited protease. In one preferred embodiment, the protease is an endopeptidase.

In some embodiments, proteases are derived from, or encoded by, a viral genome. Typically, such proteases are dependent on expression and proteolytic processing of a polyprotein and/or other events required as part of the life cycle of viruses such as Picornavirales, Nidovirales, Herpesvirales, Retroviruses and Adenoviruses, although without limitation thereto. Particular examples of proteases include: Potyviridae proteases such as the NIa protease of tobacco etch virus (TEV), tobacco vein mottling virus (TVMV), sugarcane mosaic virus (SMV) etc; Flaviviridae proteases such as the NS3 protease of hepatitis C virus (HCV); Picornaviridae proteases such as the 3C protease of EV71, Norovirus etc, the 2A protease of human rhinovirus, coxsackievirus B4 etc and the leader protease of foot and mouth disease virus (FMDV) etc; Coronaviridae proteases such as the 3C-like protease of SARS-CoV, IBV-CoV and Herpesvirus proteases such as HSV-1, HSV-2, HCMV and MCMV proteases etc, although without limitation thereto.

Preferably, the viral genome is of a plant virus. More preferably, the plant virus is a Potyvirus. In a particularly preferred embodiment, the protease is a Potyvirus protease such as the NIa protease of TEV, TVMV or SMV. In an alternative embodiment the protease is an NS3 protease of a Flavivirus such as HCV.

In other embodiments, proteases are SUMO related proteases that includes ubiquitin (Ub), NEDD8, and Atg 8 proteases. These proteases are converted into an autoinhibited form by fusion with their respective recognition domains (e.g SUMO) via a protease-resistant linker.

In an embodiment, the protease cleavage site is a TVMV cleavage site such as ETVRFQS (SEQ ID NO:32) or a functional variant thereof. The protease cleavage site may alternatively be a Thrombin cleavage site such as SEQ ID NO: 33 or a functional variant thereof, or Factor Xa site such as SEQ ID NO: 34 or SEQ ID NO: 35 or a functional variant thereof.

Examples of autoinhibited sensors responsive to protease cleavage are provided by SEQ ID NOs 39 and 40 or variants thereof. These sensors incorporate an autoinhibitory module based on inhibitory interaction between an ePDZ domain and an ePDZ peptide, separated by a protease cleavage site. Protease cleavage disrupts the inhibitory interaction and thus provides for activation of the sensor.

Variants

It will be appreciated that the biosensors and the molecular components thereof described herein may be, or comprise, contiguous amino acid sequences such as in the form of chimeric proteins or fusion proteins as are well understood in the art. Optionally, respective amino acid sequences (e.g binding moieties, enzyme amino acid sequences, protease amino acid sequences etc) may be discrete or separate amino acid sequences linked or connected by spacers or linkers (e.g. amino acids, amino acid sequences, nucleotides, nucleotide sequences or other molecules) to optimize features or activities such as target molecule recognition, binding and enzyme activity or inhibition, although without limitation thereto. Non-limiting examples of amino acid sequences inclusive of enzyme amino acid sequences, engineered mutants, linkers, protease cleavage sites, and binding moieties are provided as sequences of the invention below, as SEQ ID NOS: 1-42.

It will also be appreciated that the invention includes biosensor molecules that are variants of the embodiments described herein, or which comprise variants of the constituent protease, sensor and/or inhibitor amino acid sequences disclosed herein. Typically, such variants have at least 80%, at least 85%, preferably at least 90%, 91%, 92%, 93%, 94% 95%, 96%, 97%, 98% or 99% sequence identity with any of the amino acid sequences disclosed herein, such as SEQ ID NOS:1-36 or portions thereof. By way of example only, conservative amino acid variations may be made without an appreciable or substantial change in function. For example, conservative amino acid substitutions may be tolerated where charge, hydrophilicity, hydrophobicity, side chain “bulk”, secondary and/or tertiary structure (e.g. helicity), target molecule binding, protease activity and/or protease inhibitory activity are substantially unaltered or are altered to a degree that does not appreciably or substantially compromise the function of the biosensor. Variants of the invention (other than the engineered non-active mutants described herein) are selected to be functional and so retain or substantially retain catalytic activity (such as GDH activity), or the ability to reconstitute such catalytic activity when provided together with suitable further components of a biosensor as described above, under conditions promoting catalytic activity. Where the variant is a peptide sensor of the invention, such conditions may comprise presence or absence of the peptide. The conditions may further comprise presence of respective binding moieties, and also further comprise presence of a target molecule. Variants of the non-covalently associating amino acid sequences (such as first and second fragment sequences) described herein are selected to retain the ability to reconstitute a stable enzyme when provided in combination with their respective binding partner sequence. Variants of binding moieties described herein are selected to be functional and so retain affinity for a respective binding moiety. The binding affinity of a variant is typically sufficient that interaction between the respective binding moieties is able to regulate catalytic activity as described herein. Variants of the peptides and heterologous amino acid sequences described herein are selected to bind their respective partner (the heterologous amino acid sequence or peptide) with affinity sufficient to regulate catalytic activity as described herein. Optionally, the affinity may be cooperatively enhanced by interaction between respective binding moieties to regulate catalytic activity as described herein.

The term “sequence identity” is used herein in its broadest sense to include the number of exact amino acid matches having regard to an appropriate alignment using a standard algorithm, having regard to the extent that sequences are identical over a window of comparison. Sequence identity may be determined using computer algorithms such as GAP, BESTFIT, FASTA and the BLAST family of programs as for example disclosed by Altschul et al., 1997, Nucl. Acids Res. 25 3389. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

The variants may be functional fragments of proteins or peptides of the invention, suitably retaining their relevant catalytic activity or binding activity as applicable. Fragments are typically N- and/or C-terminal truncations. Protein fragments may comprise up to 5%, 10%, 15%, 20%, 25%, 30%, 35%, 40%, 45%, 50%, 55%, 60%, 65%, 70%, 75%, preferably up to 80%, 85%, more preferably up to 90% or up to 95-99% of an amino acid sequence disclosed herein. In some embodiments, the protein fragment may comprise up to 5, 10, 20, 40, 50, 70, 80, 90, 100, 120, 150, 180 200, 220, 230, 250, 280, 300, 330, 350, 400 or 450 amino acids of an amino acid sequence disclosed herein, such as SEQ ID NOS: 1-42.

Polypeptides Representing First and Second Oxidoreductase Fragments

In a related aspect, the invention further provides a polypeptide comprising a first fragment sequence of an oxidoreductase enzyme, preferably a glucose dehydrogenase (GDH) enzyme, which is capable of non-covalently interacting with a polypeptide comprising a second fragment sequence of said enzyme to reconstitute a stable oxidoreductase enzyme, wherein the first and second fragment sequences represent sequences obtainable by cleavage of the enzyme at a location comprising one or more residues which influence substrate binding by said enzyme. Preferably, the location comprises one or more residues which influence substrate binding and one or more residues which influence catalytic activity of said enzyme. The inventors have identified that it is possible to cleave an enzyme at a location as described above to provide soluble fragments capable of non-covalently interacting to reconstitute a stable enzyme.

In particular, they have identified that PQQ-GDH of SEQ ID NO: 1 may be cleaved at a location comprising such residues to provide soluble polypeptide fragments, including at the preferred insert location described above. The inventors have identified in particular a soluble fragment representing residues 1-330 of SEQ ID NO:1, whose solubility is indicative of autonomous folding. Reconstitution of a stable enzyme from fragments can provide a further means of controlling enzyme activity, in addition to regulation by interaction between binding moieties and by interaction with a target molecule as described above.

The first and second fragment sequences may together constitute the complete sequence of the enzyme or together constitute sufficient sequence of the enzyme to provide for a stable form of said enzyme including its catalytic domain, as described above.

The polypeptide comprising a first fragment sequence may be capable of reconstituting a stable catalytically active enzyme with said polypeptide comprising a second fragment sequence of said enzyme. In this embodiment, the polypeptide comprising a first fragment sequence of said enzyme is able to displace a corresponding fragment sequence of said enzyme which is engineered to maintain an enzyme in a catalytically inactive state from a stable enzyme complex, to restore catalytic activity.

The polypeptide comprising a first fragment sequence may alternatively comprise one or more mutations which render a stable enzyme comprising said polypeptide catalytically inactive. In an embodiment relating to GDH, the respective amino acid sequences of the enzyme may be the sequences of SEQ ID NO: 30 or a variant thereof, and SEQ ID NO: 31 or a variant thereof. The “engineered mutant” typically comprises a H144 mutation and a mutation to one or more of Q76 and D143. The mutations are selected to reduce or abolish catalytic activity of the enzyme. Preferably, H144, Q76 and D143 are each mutated. These residues may be each mutated to alanine, or alternative mutations to alanine which reduce or abolish catalytic activity can be made. The engineered mutant may comprise the sequence of SEQ ID NO: 30 or a variant thereof, incorporating one or more or all of the above mutations, and which also produces a catalytically inactive enzyme when non-covalently associated with the at least one amino acid sequence of the enzyme. The variant may comprise alternative inactivating mutations to those discussed above at positions 76, 143 and 144. Such a polypeptide (also described as an engineered polypeptide) is also able to be displaced from said stable enzyme complex to restore catalytic activity.

Also provided is an oxidoreductase enzyme, preferably a GDH enzyme which comprises both a first fragment sequence which is engineered as described above, and also a said second fragment sequence as part of a contiguous polypeptide, where the first and second fragment sequences are separated by one or more protease cleavage sites, such that protease activity allows for the engineered fragment sequence to be displaced, and a first fragment sequence capable of restoring catalytic activity to then non-covalently associate with the second fragment sequence to form a stable catalytically active enzyme.

The polypeptides described above may comprise a binding moiety capable of interacting with a respective binding moiety on a counterpart polypeptide comprising a second fragment sequence of said enzyme, wherein the interaction between the binding moieties regulates catalytic activity of the reconstituted stable glucose dehydrogenase enzyme. The interaction between the binding moieties may be regulated by binding of a target molecule. The binding moieties and corresponding target molecule may be selected from any described herein.

A polypeptide described above may further comprises a sequence inhibiting interaction of the respective binding moieties, and one or more protease cleavage sites, wherein cleavage by the protease provides for interaction between the binding moieties. The polypeptide may further comprise a sequence enhancing binding and/or cleavage efficiency of the protease. The protease cleavage site and the sequence enhancing binding and/or cleavage efficiency of the protease may be selected from any described herein.

In particular embodiments, the first and second fragment sequences described above may be derived by cleavage of a GDH enzyme in a loop or turn region of a GDH enzyme corresponding to the loop connecting beta-sheets 4 and 5 of a PQQ-GDH or in a region corresponding to residues 328 to 332 of said enzyme (such as at residue 330 of SEQ ID NO:1). The skilled person is able to identify corresponding locations in other enzymes from structural analysis and sequence alignment. A corresponding location is typically one which allows for generation of functional fragments of said enzyme which are able to reconstitute a stable enzyme.

In this aspect, the invention additionally provides a method of engineering an oxidoreductase enzyme, preferably a glucose dehydrogenase (GDH) enzyme to provide first and second fragment sequences capable of reconstituting a stable enzyme. The method comprises selecting a suitable location in the enzyme comprising residues influencing substrate binding and at which the enzyme may be cleaved to provide said first and second fragment sequences. The method typically further comprises introducing mutations into one of said sequences which render a stable enzyme reconstituted from said sequence catalytically inactive. The method may further comprise adding one or more binding moieties to said sequences which assist non-covalent association of polypeptides comprising the sequences to reconstitute a stable catalytically active enzyme.

The invention further provides a polypeptide comprising a first fragment sequence of a GDH enzyme which comprises SEQ ID NO: 30 or a variant thereof. This polypeptide may be a polypeptide capable of reconstituting a stable catalytically active GDH enzyme as described above. This polypeptide may be engineered to render a stable enzyme comprising said polypeptide catalytically inactive as described above. A catalytically inactive variant of SEQ ID NO: 30 may comprise alternative inactivating mutations to alanine at one or more of, preferably all of H144, Q76 and D143 as described above. A variant of SEQ ID NO: 30 may be a sequence which when included in a said polypeptide is capable of reconstituting a stable GDH enzyme together with a polypeptide comprising SEQ ID NO: 31.

The invention further provides a polypeptide comprising a second fragment sequence of a GDH enzyme which comprises SEQ ID NO: 31 or a variant thereof. A variant of SEQ ID NO: 31 may be a sequence which when included in a said polypeptide is capable of reconstituting a stable GDH enzyme together with a polypeptide comprising SEQ ID NO: 30 as described above.

The above polypeptides comprising SEQ ID NO: 30 or a variant thereof and SEQ ID NO: 31 or a variant thereof may further comprise one or more binding moieties selected from any described herein. Typically, a binding moiety is provided C-terminal to the sequence of SEQ ID NO: 30 or variant thereof, and N-terminal to the sequence of SEQ ID NO: 31 or variant thereof in a said polypeptide.

A polypeptide comprising SEQ ID NO: 30 or a variant thereof is also provided which further comprises two cognate (respective) binding moieties separated by one or more, such as one, two or three protease cleavage sites. The polypeptide may additionally comprise a sequence enhancing binding and/or cleavage efficiency of the protease. The cognate binding moieties interact in the absence of the protease, which interaction is then disrupted by cleavage of the protease to allow for binding of a retained binding moiety to a respective binding moiety on a further polypeptide comprising SEQ ID NO: 31 or a variant thereof, to thereby reconstitute a catalytically active GDH enzyme. The cognate binding moieties, protease cleavage sites and sequences enhancing binding and/or cleavage efficiency may be selected from any described herein.

Also provided is a GDH enzyme comprising the sequence of SEQ NO: 30 or a variant thereof, and further engineered to comprise catalytically inactivating mutations as described above, and additionally the sequence of SEQ ID NO: 31 or a variant thereof, wherein one or more protease cleavage sites are located between said sequences, such that cleavage by a protease is able to displace said polypeptide comprising the catalytically inactive sequence from said enzyme. The GDH enzyme may further comprise a binding moiety capable of interacting with a respective binding moiety on a polypeptide comprising a first fragment sequence of a GDH enzyme which comprises SEQ ID NO: 30 or a variant thereof, optionally in the presence of a target molecule, wherein interaction between the binding moieties allows for reconstitution of a stable GDH enzyme.

The above first and second fragment sequences are preferably not obtainable by cleavage of a GDH enzyme at a loop or turn region corresponding to the loop connecting beta-sheets 3A and 3B of a PQQ-GDH of SEQ ID NO: 1 or a region corresponding to residues 153-155 of said enzyme, or a corresponding region thereto.

### Biosensors

As discussed above, the oxidoreductase enzymes of the invention are particularly suitable for incorporation in biosensors, and thus the invention also provides a biosensor comprising a said enzyme. The invention also provides a biosensor comprising the first and second polypeptides representing fragment sequences described above. The biosensor may be suitable for detection of any target molecule described herein. Any suitable combinations of polypeptides and enzymes which interact together to detect a target molecule as described herein may be comprised in the biosensor. The combination may further be provided together in any in vitro context, in which detection of the target molecule is possible. The polypeptides and enzymes may be provided together in solution for detection of a target molecule.

The invention also generally relates to a biosensor comprising an enzyme and a heterologous amino acid sequence that releasably maintains said enzyme in a catalytically inactive state in the presence of a peptide, wherein the heterologous amino acid sequence binds to the peptide to switch the enzyme from a catalytically active state to a catalytically inactive state. The inventors have surprisingly identified that, in contrast to biosensor architectures previously described in which catalytic activity of an enzyme is increased upon association with a target molecule, it is possible to provide a biosensor comprising a heterologous amino acid sequence and a binding peptide therefor in which interaction between the peptide and the enzyme leads to reduction in catalytic activity, where the reduction is dose-dependent on the peptide. The biosensor may comprise any enzyme having catalytic activity for a substrate. The biosensor may be engineered such that catalytic activity is regulated by interaction between binding moieties, typically interaction between binding moieties and a target molecule, as described above.

### Other Aspects

Another aspect of the invention provides a composition or kit comprising the biosensor, oxidoreductase enzyme, or the polypeptides comprising first and second fragment sequences of any of the aforementioned aspects. The composition or kit may comprise a peptide binding the heterologous amino acid sequence of a said enzyme as described above. The composition or kit may further comprise a substrate molecule. A further aspect of the invention provides a kit or composition comprising one or more biosensors disclosed herein in combination with one or more substrate molecules.

A further aspect of the invention provides a method of detecting a target molecule, said method including the step of contacting the biosensor, oxidoreductase enzyme or polypeptides comprising first and second fragment sequences of any of the aforementioned aspects with a sample to thereby determine the presence or absence of the target molecule in the sample. Suitably, the sample is a biological sample. Biological samples may include organ samples, tissue samples, cellular samples, fluid samples or any other sample obtainable, obtained, derivable or derived from an organism or a component of the organism. The biological sample can comprise a fermentation medium, feedstock or food product such as for example, but not limited to, dairy products. In particular embodiments, the biological sample is obtainable from a mammal, preferably a human. By way of example, the biological sample may be a fluid sample such as blood, serum, plasma, urine, saliva, tears, sweat, cerebrospinal fluid or amniotic fluid, a tissue sample such as a tissue or organ biopsy or may be a cellular sample such as a sample comprising red blood cells, lymphocytes, tumour cells or skin cells, although without limitation thereto. A particular type of biological sample is a pathology sample.

Suitably, the enzyme activity of the biosensor is not substantially inhibited by components of the sample (e.g. serum proteins, metabolites, cells, cellular debris and components, naturally-occurring protease inhibitors etc).

In one embodiment, the biosensor and/or methods of use may be applicable to drug testing such as for detecting the use of illicit drugs of addiction (e.g cannabinoids, amphetamines, cocaine, heroin etc.) and/or for the detection of performance-enhancing substances in sport and/or masking agents that are typically used to avoid detection of performance-enhancing substances. This may be applicable to the detection of banned performance-enhancing substances in humans and/or other mammals such as racehorses and greyhounds that may be subjected to illicit “doping” to enhance performance.

A yet further aspect of the invention provides a method of diagnosis of a disease or condition in an organism, said method including the step of contacting the biosensor, oxidoreductase enzyme or polypeptides comprising first and second fragment sequences of any of the aforementioned aspects with a biological sample obtained from the organism to thereby determine the presence or absence of a target molecule in the biological sample, determination of the presence or absence of the target molecule facilitating diagnosis of the disease or condition. The organism may include plants and animals inclusive of fish, avians and mammals such as humans. Preferably the organism is a human. The disease or condition may be any where detection of a target molecule assists diagnosis. Non limiting examples of target molecules or analytes include blood coagulation factors such as previously described, kallikreins inclusive of PSA, matrix metalloproteinases, viral and bacterial proteases, antibodies, glucose, triglycerides, lipoproteins, cholesterol, tumour antigens, lymphocyte antigens, autoantigens and autoantibodies, drugs, salts, creatinine, blood serum or plasma proteins, pesticides, uric acid, products and intermediates of human and animal metabolism and metals. This preferred aspect of the invention may be adapted to be performed as a “point of care” method whereby determination of the presence or absence of the target molecule may occur at a patient location which is then either analysed at that location or transmitted to a remote location for diagnosis of the disease or condition.

Diagnostic aspects of the invention may also be in the form of a kit comprising one or a plurality of different biosensors capable of detecting one or a plurality of different target molecules. In this regard, a kit may comprise an array of different biosensors capable of detecting a plurality of different target molecules. The kit may further comprise one or more amplifier molecules, deactivating molecules and/or labeled substrates, as hereinbefore described. The kit may also comprise additional components including reagents such as buffers and diluents, reaction vessels and instructions for use.

A still yet further aspect of the invention provides a detection device that comprises a cell or chamber that comprises the biosensor, oxidoreductase enzyme or polypeptides comprising first and second fragment sequences of any of the aforementioned aspects. Suitably, a sample may be introduced into the cell or chamber to thereby facilitate detection of a target molecule. In certain embodiments, the detection device is capable of providing an electrochemical, acoustic and/or optical signal that indicates the presence of the target molecule.

In some embodiments, the detection device may comprise an electrode. In some embodiments the detection device may comprise a semiconductor device.

The detection device may further provide a disease diagnosis from a diagnostic target result by comprising: a processor; and

a memory coupled to the processor, the memory including computer readable program code components that, when executed by the processor,

perform a set of functions including: analysing a diagnostic test result and providing a diagnosis of the disease or condition.

The detection device may further provide for communicating a diagnostic test result by comprising: a processor; and

a memory coupled to the processor, the memory including computer readable program code components that, when executed by the processor, perform a set of functions including: transmitting a diagnostic result to a receiving device; and optionally receiving a diagnosis of the disease or condition from the or another receiving device.

The biosensor, oxidoreductase enzyme or polypeptides comprising first and second fragment sequences of any of the aspects described herein may form part of a biofuel cell. The biofuel cells may comprise the biosensor, oxidoreductase enzyme or polypeptides comprising first and second fragment sequences of any of the aspects described herein located at the anode. As described herein the biosensor or enzyme may act as an electron donor and the electrons may flow from the anode to the cathode in the biofuel cell. The biosensor or enzyme may thereby provide electrons for a chemical reaction occurring at the cathode of the biofuel cell.

The invention further provides a nucleic acid (typically in isolated form) encoding the biosensor of any of the aforementioned aspects, or any component thereof, including an oxidoreductase enzyme of the invention or a polypeptide comprising a first or second fragment sequence of the invention. The nucleic acid may encode any of SEQ ID Nos 3-12, 16, 20-21, 24-25, 27-31 and 37-40 or a variant thereof as discussed above. Another related aspect of the invention provides a genetic construct comprising the isolated nucleic acid of the aforementioned aspect. A further related aspect of the invention provides a host cell comprising the genetic construct of the aforementioned aspect. The term “nucleic acid” as used herein designates single- or double-stranded mRNA, RNA, cRNA, RNAi, siRNA and DNA inclusive of cDNA, mitochondrial DNA (mtDNA) and genomic DNA. The invention also provides variants and/or fragments of the isolated nucleic acids. Variants may comprise a nucleotide sequence at least 70%, at least 75%, preferably at least 80%, at least 85%, more preferably at least 90%, 91%, 93%, 94%, 95%, 96%, 97%, 98% or 99% nucleotide sequence identity with any nucleotide sequence disclosed herein. In other embodiments, nucleic acid variants may hybridize with the nucleotide sequence of with any nucleotide sequence disclosed herein, under high stringency conditions. Fragments may comprise or consist of up to 5%, 10%, 15%, 20%, 25%, 30%, 35%, 40%, 45%, 50%, 55%, 60%, 65%, 70%, 75%, 80%, 85%, 90% or 95-99% of the contiguous nucleotides present in any nucleotide sequence disclosed herein. Fragments may comprise or consist of up to 20, 50, 100, 150, 200, 250, 300, 350, 400, 450, 500, 550, 600, 650, 700, 750, 800, 850, 900 950, 1000, 1050, 1100, 1150, 1200, 1350 or 1300 contiguous nucleotides present in any nucleotide sequence disclosed herein.

The invention also provides “genetic constructs” that comprise one or more isolated nucleic acids, variants or fragments thereof as disclosed herein operably linked to one or more additional nucleotide sequences.

As generally used herein, a “genetic construct” is an artificially created nucleic acid that incorporates, and/or facilitates use of, an isolated nucleic acid disclosed herein.

In particular embodiments, such constructs may be useful for recombinant manipulation, propagation, amplification, homologous recombination and/or expression of said isolated nucleic acid. A still further related aspect provides a method of producing a recombinant protein biosensor or a component thereof or an oxidoreductase enzyme or GDH enzyme of the invention or a polypeptide comprising a first or second fragment sequence of a GDH enzyme, said method including the step of producing the recombinant protein biosensor or a component thereof in the host cell of the previous aspect. As used herein, a genetic construct used for recombinant protein expression is referred to as an “expression construct”, wherein the isolated nucleic acid to be expressed is operably linked or operably connected to one or more additional nucleotide sequences. in an expression vector. An “expression vector” may be either a self-replicating extra-chromosomal vector such as a plasmid, or a vector that integrates into a host genome.

The one or more additional nucleotide sequences are typically regulatory nucleotide sequences. By “operably linked” or “operably connected” is meant that said regulatory nucleotide sequence(s) is/are positioned relative to the nucleic acid to be expressed to initiate, regulate or otherwise control expression of the nucleic acid.

Regulatory nucleotide sequences will generally be appropriate for the host cell used for expression. Numerous types of appropriate expression vectors and suitable regulatory sequences are known in the art for a variety of host cells. One or more regulatory nucleotide sequences may include, but are not limited to, promoter sequences, leader or signal sequences, ribosomal binding sites, transcriptional start and termination sequences, translational start and termination sequences, splice donor/acceptor sequences and enhancer or activator sequences. Constitutive or inducible promoters as known in the art may be used and include, for example, nisin-inducible, tetracycline-repressible, IPTG-inducible, alcohol-inducible, acid-inducible and/or metal-inducible promoters. In one embodiment, the expression vector comprises a selectable marker gene. Selectable markers are useful whether for the purposes of selection of transformed bacteria (such as bla, kanR, ermB and tetR) or transformed mammalian cells (such as hygromycin, G418 and puromycin resistance).

Suitable host cells for expression may be prokaryotic or eukaryotic, such as bacterial cells inclusive of Escherichia coli (DH5a for example), yeast cells such as S. cerivisiae or Pichia pastoris, insect cells such as SF9 cells utilized with a baculovirus expression system, or any of various mammalian or other animal host cells such as CHO, BHK or 293 cells, although without limitation thereto. Introduction of expression constructs into suitable host cells may be by way of techniques including but not limited to electroporation, heat shock, calcium phosphate precipitation, DEAE dextran-mediated transfection, liposome-based transfection (e.g. lipofectin, lipofectamine), protoplast fusion, microinjection or microparticle bombardment, as are well known in the art.

Purification of the recombinant biosensor molecule may be performed by any method known in the art. In preferred embodiments, the recombinant biosensor molecule comprises a fusion partner (preferably a C-terminal His tag) which allows purification by virtue of an appropriate affinity matrix, which in the case of a His tag would be a nickel matrix or resin. The resulting, engineered mutant is preferably expressed in bacteria such as E. coli as en epitope-tagged protein and is purified by affinity chromatography.

The invention additionally provides a method of engineering an oxidoreductase enzyme, preferably a glucose dehydrogenase (GDH) enzyme comprising a heterologous amino acid sequence which is responsive to a peptide, wherein binding of the peptide to the heterologous amino acid sequence reversibly regulates catalytic activity of the enzyme. The method comprises selecting a suitable location in the enzyme able to tolerate insertion of the heterologous amino acid sequence, typically a location comprising residues influencing substrate binding by and/or catalytic activity of said enzyme as described above, and inserting said heterologous amino acid sequence into the enzyme, such that an enzyme is engineered which responds to the peptide to regulate catalytic activity of the enzyme.

So that the invention may be readily understood and put into practical effect, embodiments of the invention will be described with reference to the following non-limiting Examples.

### EXAMPLES

### Materials and Methods

### Chimeric Gene Construction and Protein Expression and Purification

The constructs of GDH-CaM or GDH-PDZ chimeric proteins were generated by Gibson Assembly™ method according the manufacturer instruction (New England Biolab) and cloned into PET28a vector. The gene fragments for the assemble were made either by PCR or by Gblcok gene synthesis from IDT (Integrated DNA Technologies). The protein expression and purification were described by Olsthoorn & Duine¹⁹. The purified GDH-CaM were reconstituted by adding PQQ with 1:1.5 ratio. This ratio for reconstitution of GDH and PQQ was also used in all other experiments using PQQ-GDH enzymes described herein.

The proteins of cyclosporine sensor were purified as described previously (http://www.pnas.org/content/99/21/13522). After Ni-NTA purification the pooled enzyme-containing fractions were supplemented with EDTA to the final concentration 5 mM and dialyzed against buffer containing 20 mM KH₂PO₄ pH7.0 and 5 mM EDTA for 10 hours. Subsequently EDTA was removed by dialyzing the sample against the buffer containing 20 mM KH₂PO₄ pH7.0 only.

### Analysis of GDH Enzymatic Activity

The GDH enzyme assay was performed as described by Yu et al.²⁰ Briefly, the 1.5-mL assay system consisted of 20 mM glucose, 0.6 mM phenazine methosulfate, 0.06 mM 2,6-dichlorophenol, 10 mM MOPS (pH 7.0), and corresponding concentration of CaCl₂ and enzyme. The enzymatic assay was performed at 25° C. by monitoring the reduction in the absorbance of 2,6-dichlorophenol at 600 nm.

### Example 1—Rationale for Construction of Insertion Peptide-Responsive Mutant of PQQ-GDH

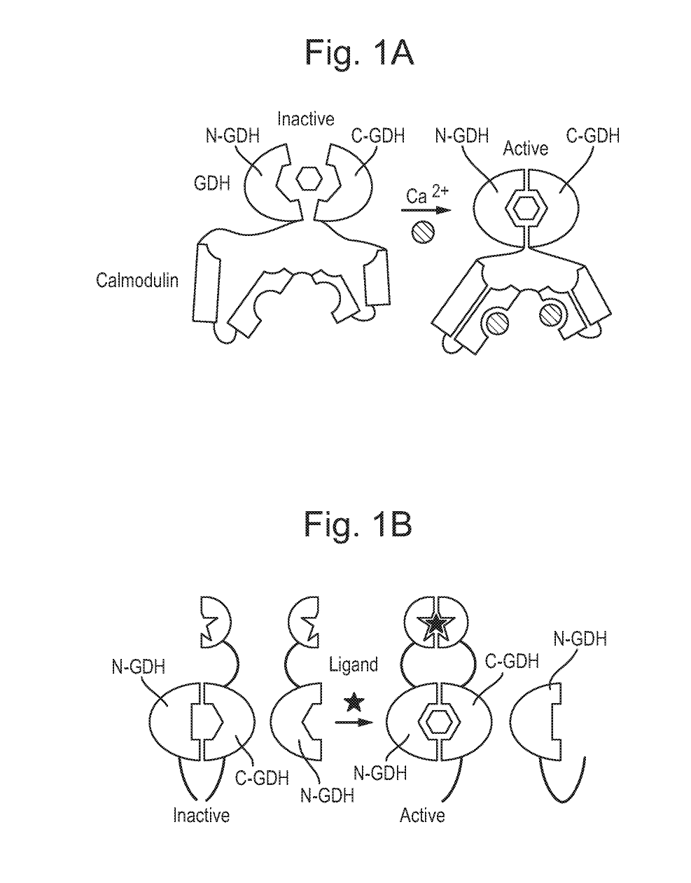

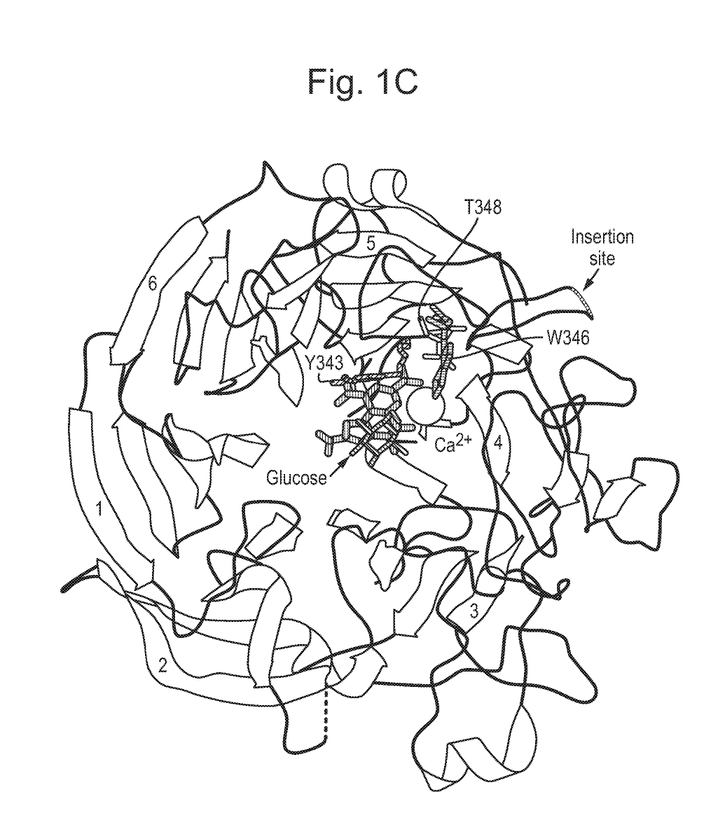

We previously identified the loop connecting strands A and B of beta-sheet 3 of PQQ-GDH as a site able to tolerate insertion of a calmodulin protein. The resulting calmodulin-GDH chimera (FIG. 1A) demonstrated calcium-binding activity and acted solely as a calcium sensor. The same location was also amenable to splitting GDH into two inactive fragments that could then be reconstituted into an active enzyme via scaffolding interactions with an analyte (FIG. 1B, SEQ ID NOs 43 and 44). The splitting approach allowed for coupling of GDH activity to detection of various analytes, but required proteolytic cleavage and purification of components, and also had a response rate governed by the rate of reconstitution of components. Analyte-drived dimerisation was also required for activity.

We sought an approach that would allow conversion of GDH into a allosteric peptide regulated ON or OFF switch that can be subsequently integrated into more complex receptor architectures. We identified a loop connecting (3-sheets 4 and 5 as a suitable insertion site as it is harboring Trp346 and Tyr348 that form a part of glucose binding site. The same loop also includes Thr348 that is involved in coordination of PQQ in the active site⁶ (FIG. 1C).

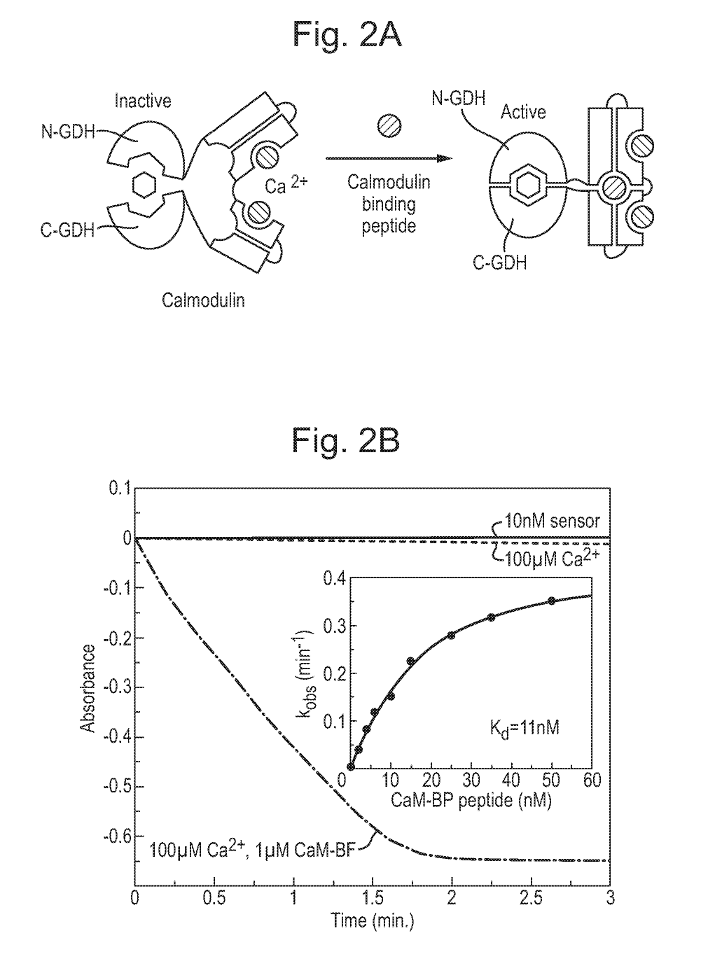

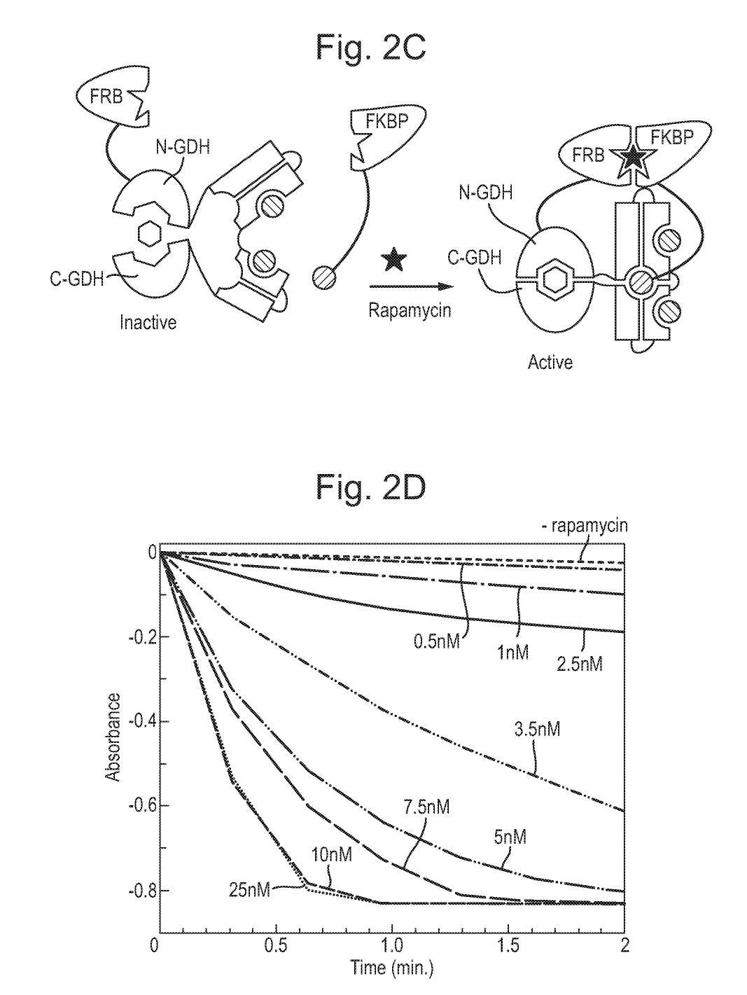

We conjectured that dislocation of either of these residues will impact the catalysis and if carried out in a reversible fashion could utilized for controlling enzymes activity. To test this idea we inserted calmodulin (CaM) domain into GDH at the position 330 and produced the resulting CaM-GDH chimeric protein (SEQ ID NO: 10) in recombinant form. The resulting protein displayed some GDH activity that was potently suppressed by addition of Ca²⁺ (FIG. 2A). However, addition of calmodulin binding peptide (CaM-BP, SEQ ID NO: 4) extracted from crystal structure of CaM:CBP complex (PDB:2BBM) increased the GDH activity of the chimera in dose dependent fashion. The activity of fully activated fusion represented 50% of activity of the wild-type protein. Fit of the observed reaction rates showed that peptide bound to the enzyme with the affinity of 11 nM indicating that binding was affinity was decreased due to the by the chimeric nature of the protein. We therefore concluded that we successfully constructed a peptide regulated GDH biosensor module.

### Example 2-Construction of Two Component Biosensors Based on the Peptide-Activated GDH Chimera

We next decided to test if the developed allosteric module can be used to construct a generic biosensor architecture. To this end we fused the developed CaM-GDH chimera C-terminally with rapamycin-binding FKBP domain (SEQ ID NO: 11) and produced the protein in recombinant form. As expected the protein displayed minimal GDH activity in the absence of the CaM-BP. We then constructed a fusion between FKBP binding partner FRB and CaM-BP that would associate with the former reporter molecule in the rapamycin dependent fashion. We reasoned that such a unit should operate cooperatively and its overall affinity in the absence of the ligand should be at least an order of magnitude lower than in its presence.

Therefore we analyzed the structure of CaM:CaM-BP complex (PDB;2BBM) to design a mutation of CaM-BP that would on one hand significantly reduce the affinity of the CaM-BP. We concluded that truncating the ligand peptide by 16 amino acids and replacing last 5 amino acids with ASASA sequence (SEQ ID NO: 8) would on one hand reduce the affinity of the peptide for the CaM but on the other hand preserve enough structural contacts that a CaM:CaM-BP complex could be formed. This is in line with the recent observation that CaM is capable of accommodating a broad range of peptide substrates when they are present at high concentrations⁷.

Mixing the solutions of CaM-GDH-FKBP and FRB-CaM-BP (SEQ ID NO 12) induced only a low level of GDH activity. However, addition of rapamycin rapidly and dose dependently induced GDH activity allowing determination of the concentration and Kd of the compound.

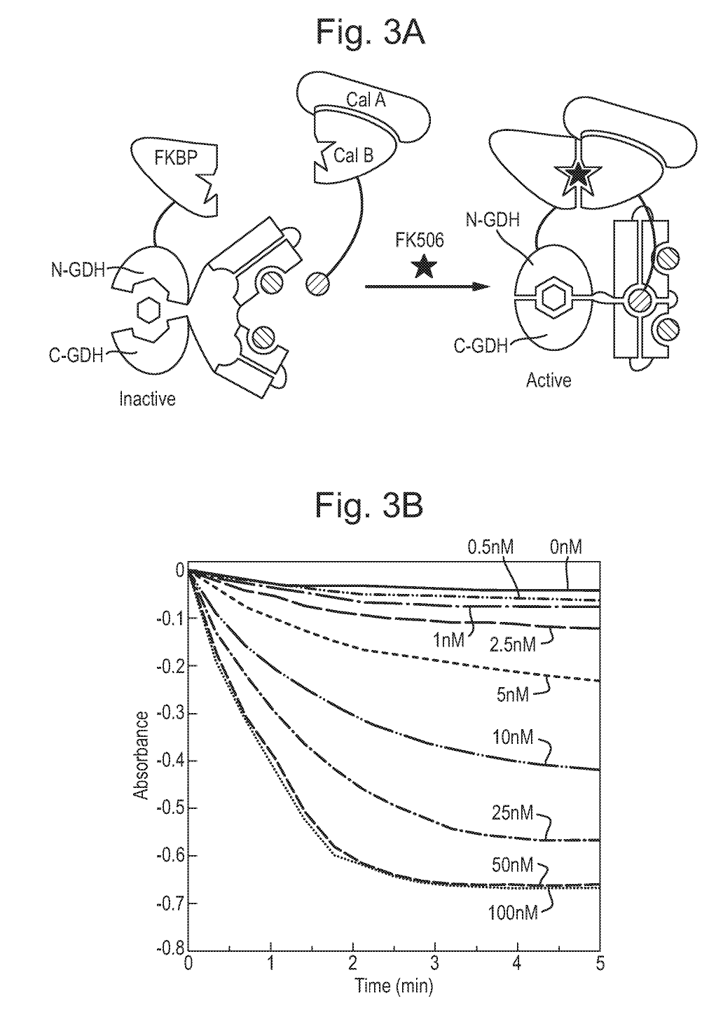

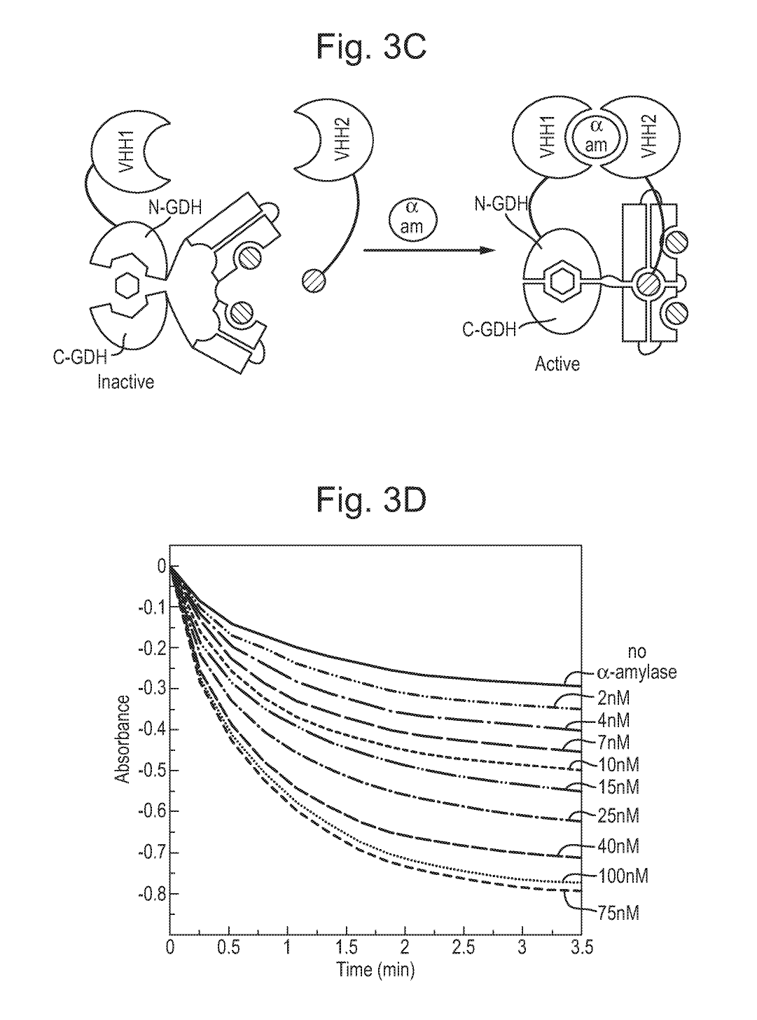

As dimerization is the driving force in biosensor activation we expected the developed architecture to be generic. To test that we set out to construct a biosensor of another immunosuppressant drug FK506. For that we fused the developed GDH-CaM chimera to FKBP12 (SEQ ID NO: 11) while the modified CaM-BP was fused to the calcineurin B (SEQ ID NO: 16). The latter was co-expressed in E. coli together with calcineurin A fused to SUMO protein (SEQ ID NO: 15) and purified as a complex using Ni-NTA resin and followed by size exclusion chromatography. When the solution of the both biosensor components was titrated with FK506 we detected a dose dependent increase in GDH activity (FIG. 3B,C) demonstrating that biosensors of small molecules other than rapamycin could be constructed using the developed biosensor architecture.

We next tested if the approach could be applied to detection of proteins rather than small molecules and produced fusions of Cam-GDH and modified version of CaM-BP in fusion with VHH domains (SEQ ID Nos 20 and 21) targeting two different epitopes of α-amylase⁸. Addition of a—amylase to the solution of these fusion proteins led to a dose-dependent increase in GDH activity indicating that the architecture is generic (FIG. 3C,D).

### Example 3—Construction of a Ligand-Activated Sterically Auto-Inhibited Cam-GDH Module

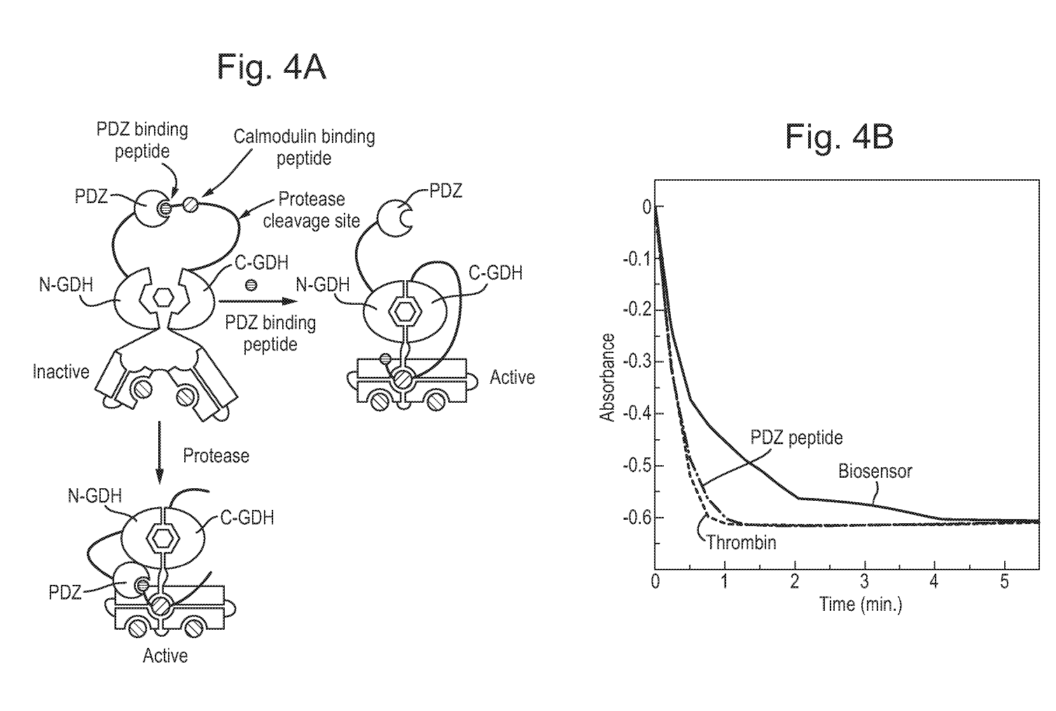

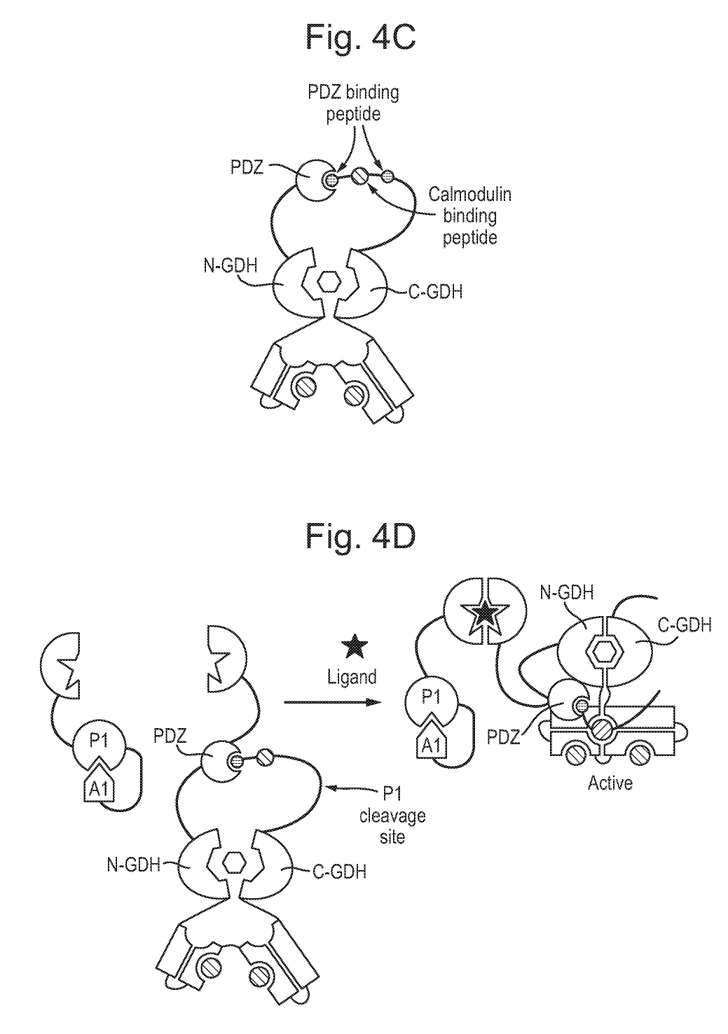

Next we attempted to aggregate the developed biosensor architecture into a single sensory unit. We conjectured that if the activating peptide could be kept away from the Cam-GDH in the ligand controlled fashion it would allow both parts of the biosensor to reside in the same molecule. To this end we constructed a fusion protein consisting of Cam-GDH chimera flanked by the PDZ domain and a fusion of CaM-BP fused to PDZ domain binding peptide via thrombin cleavage site (FIG. 4A, SEQ ID NO: 29). The resulting fusion protein displayed reduced GDH activity that could be induced by the exposure to the PDZ peptide or thrombin protease (FIG. 4B).

Further improvements of the biosensors could include additional binding sites for steric inhibitor that would shift the equilibrium towards the sterically auto inhibited state (FIG. 4C). While the presented example is based on detection of PDZ peptide any other binder ligand pair could be used, such as antibody/antigen, small molecule binding domain, protein:DNA, or protein:RNA.

In a further embodiment the autoinhibited module could be integrated into a two component biosensor architecture where the auto-inhibited module is activated by a protease brought into proximity through scaffolding interactions (FIG. 4D). The protease can be constitutively active or auto-inhibited thereby reducing the background activation.

### Example 4—Developing an OFF GDH-Based Biosensor

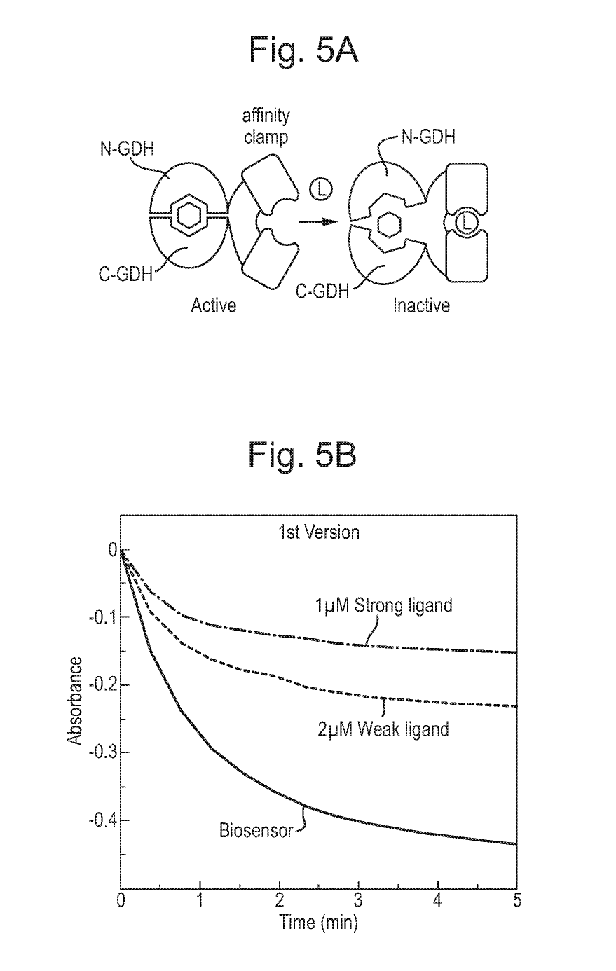

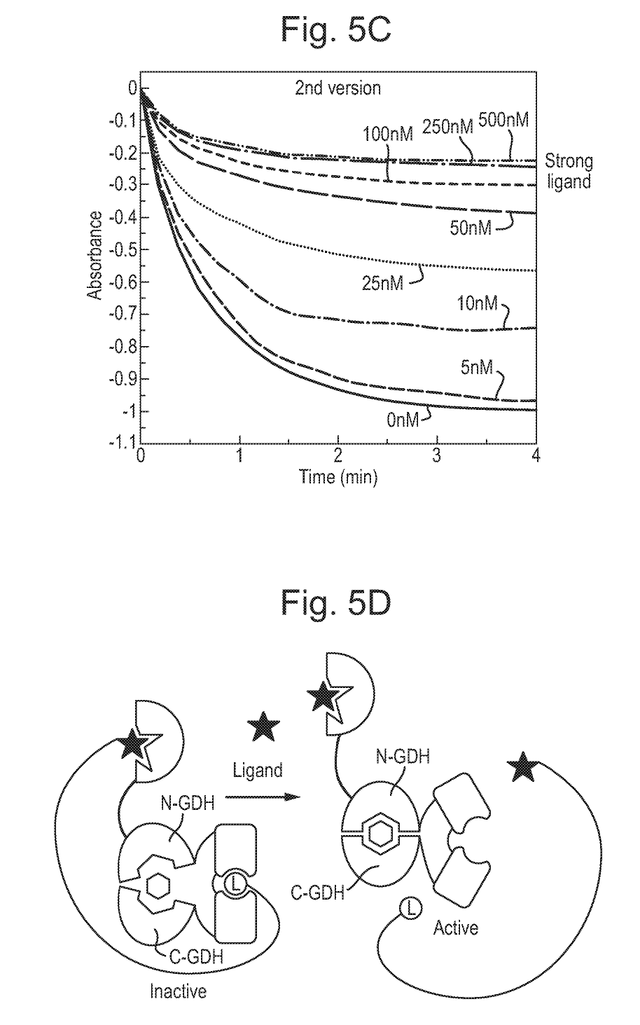

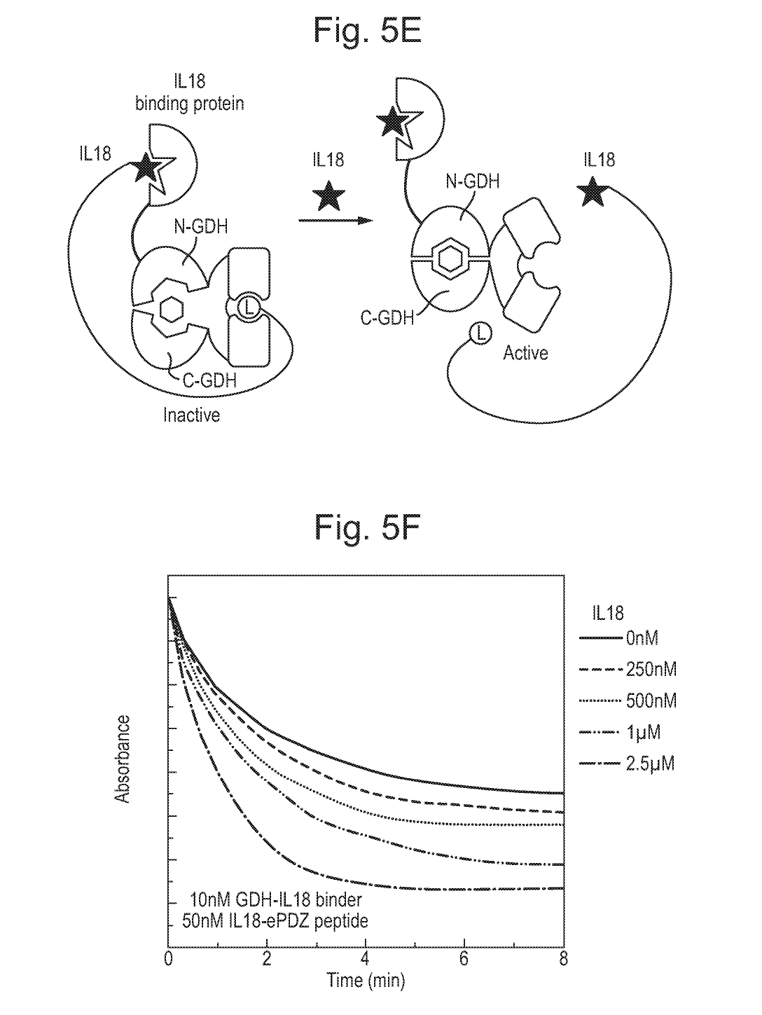

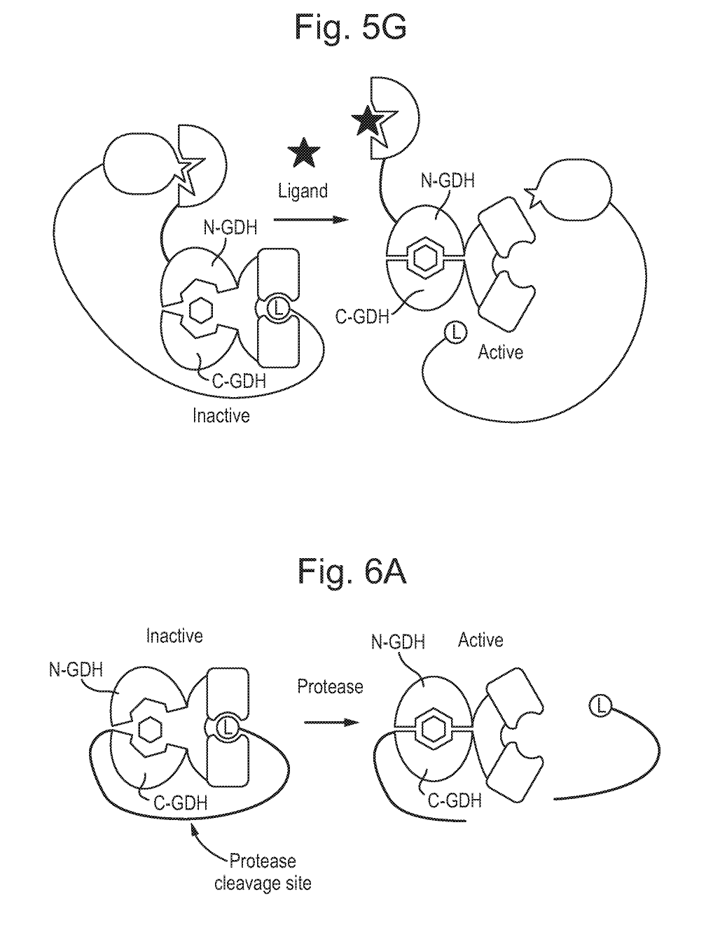

So far all presented biosensor architectures were designed to increase the GDH activity upon association with the analyte. While this is a common strategy it creates potential problems when binding of two protein modules to a single small molecule is required. We therefore decided to exploit an alternative architecture where the dissociation of the complex would lead to increase in GDH activity. This would require development of an inhibitory GDH:ligand pair. To achieve that we chose use of an “affinity clamp”—an artificial two domain receptor composed of a circularly permutated Erbin PDZ domain connected by a flexible serine-glycine linker to an engineered fibronectin type III (FN3)⁹. This module was shown to bind a PDZ domain binding peptide with affinities below 1 nM and undergo large conformational transitions upon ligand binding^{10,11}. We inserted into the loop connecting (3-sheets 4 and 5 of GDH and produced the protein in the recombinant form (FIG. 5A, SEQ ID NO: 25).

When the solution of recombinant biosensor was exposed to a solution of PDZ peptides the GDH activity was markedly inhibited (FIG. 5B). There was a clear correlation between the affinity of the ligand and the extent of the inhibition with high affinity ligand (SEQ ID NO: 27) inducing stronger inhibition of GDH activity than the weaker ligand (SEQ ID NO: 28). It is well established that linkers connecting the sensory domain and actuators play a critical role in performance of biosensors^{12,13}. We therefore reanalyzed the model of both proteins and optimized the linkers on the both side of the affinity clamp sequence. The resulting biosensor showed both increased response to the ligands and more rapid reaction kinetics (FIG. 5C).

The developed GDH-based OFF switch can be converted into biosensors of small molecules and biological polymers such as proteins and nucleic acids by fusing it to the appropriate binding domains (FIG. 5 D) FIG. 5E depicts an IL18 biosensor that was generated incorporating a GDH-based OFF switch based on a fusion with IL18 binding protein, where the ligand is IL18 and the binder IL18 binding protein. This biosensor provided for dose-dependent detection of IL18 as shown in FIG. 5F. A further alternative configuration of the OFF switch is shown in FIG. 5G.

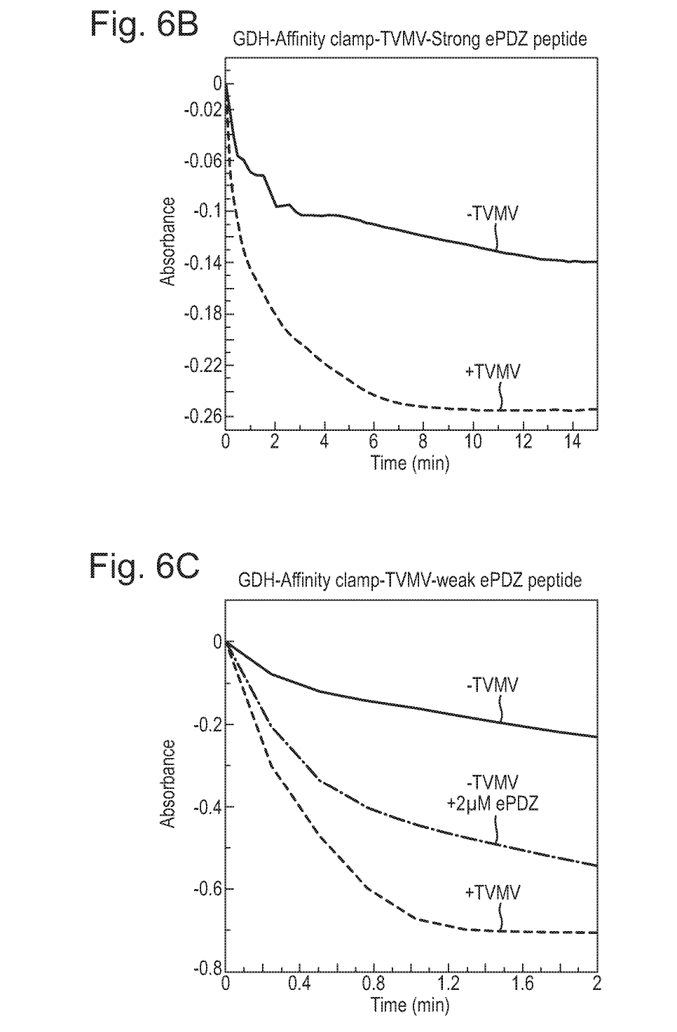

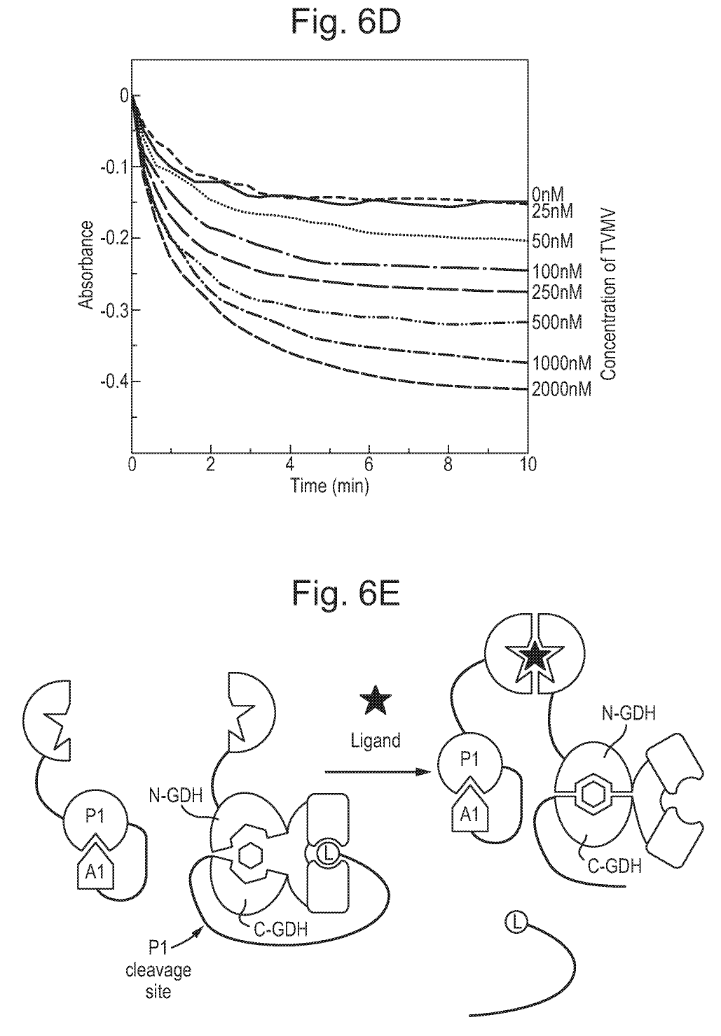

The GDH-based OFF switch can also be converted into an autoinhibited protease biosensor, activatable by protease cleavage. This module is shown in FIG. 6A, with activity data for different variants following protease cleavage as shown in FIGS. 6B-6D. FIG. 6E illustrates how the autoinhibited protease biosensor could be subsequently integrated in to the ultrasensitive two component architecture.

### Example 5—Comparison of Sensitivity of New Architecture with Previous Split Architecture

We compared the activation rates of 10 nM of a split enzyme as described in Example 1, SEQ ID NOs 43 and 44), and a modified CaM insert with optimised linker rapamycin biosensor. The split biosensor was based on 10 nM GDH with the TVMV cleavage site in the loop connecting strands A and B and carrying mutations Gln76Ala, Asp143 Ala, and His144 Ala in the active site fused C-terminally to FKBP, 15 nM 1-153 fragment of GDH fused to N-terminus of FRB. The insert biosensor was based on 10 nM GDH-CalM-FKBP, 2.5 μM FRB-CalM BP. Assays were carried out with 50 μM CaCl₂), 0.6 mM PMS, 0.06 mM DCPIP, 20 mM Glucose.

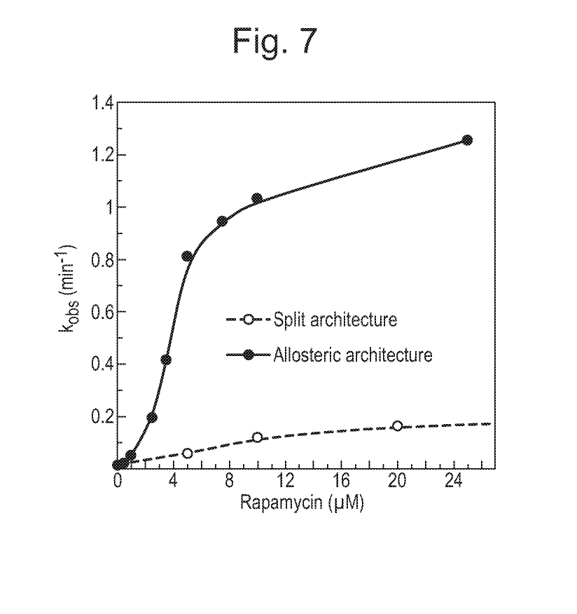

The data shown in FIG. 7 demonstrated that the insert biosensor had both faster rate of response and higher total electron yield. Therefore use of a GDH-CaM chimera-based biosensor is improved over the split architecture. Furthermore preparation of the GDH-CaM chimera-based biosensor did not require proteolytic cleavage of the precursor protein making their preparation straightforward.

Throughout the specification, the aim has been to describe the preferred embodiments of the invention without limiting the invention to any one embodiment or specific collection of features. Various changes and modifications may be made to the embodiments described and illustrated without departing from the present invention.

The disclosure of each patent and scientific document, computer program and algorithm referred to in this specification is incorporated by reference in its entirety.

SEQUENCES OF THE INVENTION

mature PQQ-GDH protein (SEQ ID NO: 1)
DVPLIPSQFAKAKSENFDKKVILSNLNKPHALLWGPDNQIWLTERATGKILRVN
PESGSVKTVFQVPEIVNDADGQNGLLGFAFHPDFKNNPYIYISGTFKNPKSTDKE
LPNQTIIRRYTYNKSTDTLEKPVDLLAGLPSSKDHQSGRLVIGPDQKIYYTIGDQ
GRNQLAYLFLPNQAQHTPTQQELNGKDYHTYMGKVLRLNLDGSIPKDNPSFNG
VVSHIYTLGHRNPQGLAFTPNGKLLQSEQGPNSDDEINLIVKGGNYGWPNVAG
YKDDSGYAYANYSAAANKTIKDLAQNGVKVAAGVPVTKESEWTGKNFVPPL
KTLYTVQDTYNYNDPTCGEMTYICWPTVAPSSAYVYKGGKKAITGWENTLLV
PSLKRGVIFRIKLDPTYSTTYDDAVPMFKSNNRYRDVIASPDGNVLYVLTDTAG
NVQKDDGSVTNTLENPGSLIKFTYKAK
Protein sequence before cleavage of signal sequence (SEQ ID NO: 2)
MNKHLLAKIALLGAAQLVTLSAFADVPLIPSQFAKAKSENFDKKVILSNLNKPH
ALLWGPDNQIWLTERATGKILRVNPESGSVKTVFQVPEIVNDADGQNGLLGFA
FHPDFKNNPYIYISGTFKNPKSTDKELPNQTIIRRYTYNKSTDTLEKPVDLLAGLP
SSKDHQSGRLVIGPDQKIYYTIGDQGRNQLAYLFLPNQAQHTPTQQELNGKDY
HTYMGKVLRLNLDGSIPKDNPSFNGVVSHIYTLGHRNPQGLAFTPNGKLLQSEQ
GPNSDDEINLIVKGGNYGWPNVAGYKDDSGYAYANYSAAANKTIKDLAQNGV
KVAAGVPVTKESEWTGKNFVPPLKTLYTVQDTYNYNDPTCGEMTYICWPTVA
PSSAYVYKGGKKAITGWENTLLVPSLKRGVIFRIKLDPTYSTTYDDAVPMFKSN
NRYRDVIASPDGNVLYVLTDTAGNVQKDDGSVTNTLENPGSLIKFTYKAK
Calmodulin protein (SEQ ID NO: 3)
TEEQIAEFKEAFSLFDKDGDGTITTKELGTVMRSLGQNPTEAELQDMINEVDAD
GNGTIDFPEFLTMMARKMKDTDSEEEIREAFRVFDKDGNGYISAAELRHVMTN
LGEKLTDEEVDEMIREADIDGDGQVNYEEFVQMMTA
CaM-binding peptide from structure PDB:2BBM (SEQ ID NO: 4)
KRRWKKNFIAVSAANRFKKISSSGAL
Modified CaM-BPs
KRRWKKNFIAVSAANRFKKIS (SEQ ID NO: 5)
KRRWKKNFIAVSAANR (SEQ ID NO: 6)
KRRWKKNFIA (SEQ ID NO: 7)
Preferred Modified CaM-BP
KRRWKKNFIAVASASA (SEQ ID NO: 8)
GDH-CaM (first generation) (SEQ ID NO: 9)
DVPLIPSQFAKAKSENFDKKVILSNLNKPHALLWGPDNQIWLTERATGKILRVN
PESGSVKTVFQVPEIVNDADGQNGLLGFAFHPDFKNNPYIYISGTFKNPKSTDKE
LPNQTIIRRYTYNKSTDTLEKPVDLLAGLPSSKDHQSGRLVIGPDQKIYYTIGDQ
GRNQLAYLFLPNQAQHTPTQQELNGKDYHTYMGKVLRLNLDGSIPKDNPSFNG
VVSHIYTLGHRNPQGLAFTPNGKLLQSEQGPNSDDEINLIVKGGNYGWPNVAG
YKDDSGYAYANYSAAANKTIKDLAQNGVKVAAGVPVTKESEWTGKNFVPPL
KTLYTVQDGSGSGGSGTEEQIAEFKEAFSLFDKDGDGTITTKELGTVMRSLGQN
PTEAELQDMINEVDADGNGTIDFPEFLTMMARKMKDTDSEEEIREAFRVFDKD
GNGYISAAELRHVMTNLGEKLTDEEVDEMIREADIDGDGQVNYEEFVQMMTA
GYNYNDPTCGEMTYICWPTVAPSSAYVYKGGKKAITGWENTLLVPSLKRGVIF
RIKLDPTYSTTYDDAVPMFKSNNRYRDVIASPDGNVLYVLTDTAGNVQKDDGS
VTNTLENPGSLIKFTYKAKHHHHHH
GDH-CaM (second generation) (SEQ ID NO: 10)
DVPLIPSQFAKAKSENFDKKVILSNLNKPHALLWGPDNQIWLTERATGKILRVN
PESGSVKTVFQVPEIVNDADGQNGLLGFAFHPDFKNNPYIYISGTFKNPKSTDKE
LPNQTIIRRYTYNKSTDTLEKPVDLLAGLPSSKDHQSGRLVIGPDQKIYYTIGDQ
GRNQLAYLFLPNQAQHTPTQQELNGKDYHTYMGKVLRLNLDGSIPKDNPSFNG
VVSHIYTLGHRNPQGLAFTPNGKLLQSEQGPNSDDEINLIVKGGNYGWPNVAG
YKDDSGYAYANYSAAANKTIKDLAQNGVKVAAGVPVTKESEWTGKNFVPPL
KTLYTVQDGSGGTEEQIAEFKEAFSLFDKDGDGTITTKELGTVMRSLGQNPTEA
ELQDMINEVDADGNGTIDFPEFLTMMARKMKDTDSEEEIREAFRVFDKDGNGY
ISAAELRHVMTNLGEKLTDEEVDEMIREADIDGDGQVNYEEFVQMMTAGGSG
GYNYNDPTCGEMTYICWPTVAPSSAYVYKGGKKAITGWENTLLVPSLKRGVIF
RIKLDPTYSTTYDDAVPMFKSNNRYRDVIASPDGNVLYVLTDTAGNVQKDDGS
VTNTLENPGSLIKFTYKAKHHHHHH
GDH-CaM-FKBP12 (SEQ ID NO: 11)
DVPLIPSQFAKAKSENFDKKVILSNLNKPHALLWGPDNQIWLTERATGKILRVN
PESGSVKTVFQVPEIVNDADGQNGLLGFAFHPDFKNNPYIYISGTFKNPKSTDKE
LPNQTIIRRYTYNKSTDTLEKPVDLLAGLPSSKDHQSGRLVIGPDQKIYYTIGDQ
GRNQLAYLFLPNQAQHTPTQQELNGKDYHTYMGKVLRLNLDGSIPKDNPSFNG
VVSHIYTLGHRNPQGLAFTPNGKLLQSEQGPNSDDEINLIVKGGNYGWPNVAG
YKDDSGYAYANYSAAANKTIKDLAQNGVKVAAGVPVTKESEWTGKNFVPPL
KTLYTVQDGSGGTEEQIAEFKEAFSLFDKDGDGTITTKELGTVMRSLGQNPTEA
ELQDMINEVDADGNGTIDFPEFLTMMARKMKDTDSEEEIREAFRVFDKDGNGY
ISAAELRHVMTNLGEKLTDEEVDEMIREADIDGDGQVNYEEFVQMMTAGGSG
GYNYNDPTCGEMTYICWPTVAPSSAYVYKGGKKAITGWENTLLVPSLKRGVIF
RIKLDPTYSTTYDDAVPMFKSNNRYRDVIASPDGNVLYVLTDTAGNVQKDDGS
VTNTLENPGSLIKFTYKAKGGSGGGVQVETISPGDGRTFPKRGQTCVVHYTGM
LEDGKKFDSSRDRNKPFKFMLGKQEVIRGWEEGVAQMSVGQRAKLTISPDYA
YGATGHPGIIPPHATLVFDVELLKLEKLAAALEHHHHHH
FRB-CaM-BP (SEQ ID NO: 12)
AHHHHHHSSGTRVAILWHEMWHEGLEEASRLYFGERNVKGMFEVLEPLHAM
MERGPQTLKETSFNQAYGRDLMEAQEWCRKYMKSGNVKDLTQAWDLYYHV
FRRISGGSGGSGSGSGGSGGKRRWKKNFIAVASASA
FKPB12 (SEQ ID NO: 13)
GVQVETISPGDGRTFPKRGQTCVVHYTGMLEDGKKFDSSRDRNKPFKFMLGKQ
EVIRGWEEGVAQMSVGQRAKLTISPDYAYGATGHPGIIPPHATLVFDVELLKLE
FRB (SEQ ID NO: 14)
LWHEMWHEGLEEASRLYFGERNVKGMFEVLEPLHAMMERGPQTLKETSFNQ
AYGRDLMEAQEWCRKYMKSGNVKDLTQAWDLYYHVFRRIS
SUMO-CN alpha subunit (SEQ ID NO: 15)
MGSSHHHHHHGSDSEVNQEAKPEVKPEVKPETHINLKVSDGSSEIFFKIKKTTPL
RRLMEAFAKRQGKEMDSLRFLYDGIRIQADQTPEDLDMEDNDIIEAHREQIGGT
SEPKAIDPKLSTTDRVVKAVPFPPSHRLTAKEVFDNDGKPRVDILKAHLMKEGR
LEESVALRIITEGASILRQEKNLLDIDAPVTVCGDIHGQFFDLMKLFEVGGSPAN
TRYLFLGDYVDRGYFSIECVLYLWALKILYPKTLFLLRGNHECRHLTEYFTFKQ
ECKIKYSERVYDACMDAFDCLPLAALMNQQFLCVHGGLSPEINTLDDIRKLDRF
KEPPAYGPMCDILWSDPLEDFGNEKTQEHFTHNTVRGCSYFYSYPAVCEFLQH
NNLLSILRAHEAQDAGYRMYRKSQTTGFPSLITIFSAPNYLDVYNNKAAVLKYE
NNVMNIRQFNCSPHPYWLPNFMDVFTWSLPFVGEKVTEMLVNVLNICSDDELG
SEEDGFDGATAAARLVTAGLVLA
CN beta subunit-CalM peptide (SEQ ID NO: 16)
DGHHHHHHGGNEASYPLEMCSHFDADEIKRLGKRFKKLDLDNSGSLSVEEFMS
LPELQQNPLVQRVIDIFDTDGNGEVDFKEFIEGVSQFSVKGDKEQKLRFAFRIYD
MDKDGYISNGELFQVLKMMVGNNLKDTQLQQIVDKTIINADKDGDGRISFEEF
CAVVGGLDIHKKMVVDVGGSGGSGSGSGGSGGKRRWKKNFIAVASASA
SUMO (SEQ ID NO: 17)
MGSSHHHHHHGSDSEVNQEAKPEVKPEVKPETHINLKVSDGSSEIFFKIKKTTPL
RRLMEAFAKRQGKEMDSLRFLYDGIRIQADQTPEDLDMEDNDIIEAHREQIGG
Calcineurin (CN) alpha subunit (SEQ ID NO: 18)
TSEPKAIDPKLSTTDRVVKAVPFPPSHRLTAKEVFDNDGKPRVDILKAHLMKEG
RLEESVALRIITEGASILRQEKNLLDIDAPVTVCGDIHGQFFDLMKLFEVGGSPA
NTRYLFLGDYVDRGYFSIECVLYLWALKILYPKTLFLLRGNHECRHLTEYFTFK
QECKIKYSERVYDACMDAFDCLPLAALMNQQFLCVHGGLSPEINTLDDIRKLD
RFKEPPAYGPMCDILWSDPLEDFGNEKTQEHFTHNTVRGCSYFYSYPAVCEFLQ
HNNLLSILRAHEAQDAGYRMYRKSQTTGFPSLITIFSAPNYLDVYNNKAAVLKY
ENNVMNIRQFNCSPHPYWLPNFMDVFTWSLPFVGEKVTEMLVNVLNICSDDEL
GSEEDGFDGATAAARLVTAGLVLA
Calcineurin (CN) beta subunit (SEQ ID NO: 19)
NEASYPLEMCSHFDADEIKRLGKRFKKLDLDNSGSLSVEEFMSLPELQQNPLVQ
RVIDIFDTDGNGEVDFKEFIEGVSQFSVKGDKEQKLRFAFRIYDMDKDGYISNG
ELFQVLKMMVGNNLKDTQLQQIVDKTIINADKDGDGRISFEEFCAVVGGLDIH
KKMVVDV
GDH-CaM-VHH1 (SEQ ID NO: 20)
DVPLIPSQFAKAKSENFDKKVILSNLNKPHALLWGPDNQIWLTERATGKILRVN
PESGSVKTVFQVPEIVNDADGQNGLLGFAFHPDFKNNPYIYISGTFKNPKSTDKE
LPNQTIIRRYTYNKSTDTLEKPVDLLAGLPSSKDHQSGRLVIGPDQKIYYTIGDQ
GRNQLAYLFLPNQAQHTPTQQELNGKDYHTYMGKVLRLNLDGSIPKDNPSFNG
VVSHIYTLGHRNPQGLAFTPNGKLLQSEQGPNSDDEINLIVKGGNYGWPNVAG
YKDDSGYAYANYSAAANKTIKDLAQNGVKVAAGVPVTKESEWTGKNFVPPL
KTLYTVQDGSGGTEEQIAEFKEAFSLFDKDGDGTITTKELGTVMRSLGQNPTEA
ELQDMINEVDADGNGTIDFPEFLTMMARKMKDTDSEEEIREAFRVFDKDGNGY
ISAAELRHVMTNLGEKLTDEEVDEMIREADIDGDGQVNYEEFVQMMTAGGSG
GYNYNDPTCGEMTYICWPTVAPSSAYVYKGGKKAITGWENTLLVPSLKRGVIF
RIKLDPTYSTTYDDAVPMFKSNNRYRDVIASPDGNVLYVLTDTAGNVQKDDGS
VTNTLENPGSLIKFTYKAKGSGSGGQVQLVESGGGTVPAGGSLRLSCAASGNTL
CTYDMSWYRRAPGKGRDFVSGIDNDGTTTYVDSVAGRFTISQGNAKNTAYLQ
MDSLKPDDTAMYYCKPSLRYGLPGCPIIPWGQGTQVTVSS
KLAAALEHHHHHH
VHH2-CaM-BP (SEQ ID NO: 21)
DGHHHHHHGSGDTTVSEPAPSCVTLYQSWRYSQADNGCAETVTVKVVYEDDT
EGLCYAVAPGQITTVGDGYIGSHGHARYLARCLGGSGGSGSGSGGSGGKRRW
KKNFIAVASASA
VHH1 (SEQ ID NO: 22)
QVQLVESGGGTVPAGGSLRLSCAASGNTLCTYDMSWYRRAPGKGRDFVSGID
NDGTTTYVDSVAGRFTISQGNAKNTAYLQMDSLKPDDTAMYYCKPSLRYGLP
GCPIIPWGQGTQVTVSS
VHH2 (SEQ ID NO: 23)
DTTVSEPAPSCVTLYQSWRYSQADNGCAETVTVKVVYEDDTEGLCYAVAPGQI
TTVGDGYIGSHGHARYLARCL
GDH-ePDZ peptide sensor first version (SEQ ID NO: 24)
DVPLIPSQFAKAKSENFDKKVILSNLNKPHALLWGPDNQIWLTERATGKILRVN
PESGSVKTVFQVPEIVNDADGQNGLLGFAFHPDFKNNPYIYISGTFKNPKSTDKE
LPNQTIIRRYTYNKSTDTLEKPVDLLAGLPSSKDHQSGRLVIGPDQKIYYTIGDQ
GRNQLAYLFLPNQAQHTPTQQELNGKDYHTYMGKVLRLNLDGSIPKDNPSFNG
VVSHIYTLGHRNPQGLAFTPNGKLLQSEQGPNSDDEINLIVKGGNYGWPNVAG
YKDDSGYAYANYSAAANKTIKDLAQNGVKVAAGVPVTKESEWTGKNFVPPL
KTLYTVQDEDAPESPELGFSISGGVGGRGNPFRPDDDGIFVTRVQPEGPASKLLQ
PGDKIIQANGYSFINIEHGQAVSLLKTFQNTVELIIVREVGNGAKQEIRVRVEKD
GGSGGVSSVPTNLEVVAATPTSLLISWDAYRELPVSYYRITYGETGGNSPVQEF
TVPGSKSTATISGLKPGVDYTITVYAHYNYHYYSSPISINYRGPGYNYNDPTCGE
MTYICWPTVAPSSAYVYKGGKKAITGWENTLLVPSLKRGVIFRIKLDPTYSTTY
DDAVPMFKSNNRYRDVIASPDGNVLYVLTDTAGNVQKDDGSVTNTLENPGSLI
KFTYKAKHHHHHH
GDH-ePDZ peptide sensor second version (SEQ ID NO: 25)
DVPLIPSQFAKAKSENFDKKVILSNLNKPHALLWGPDNQIWLTERATGKILRVN
PESGSVKTVFQVPEIVNDADGQNGLLGFAFHPDFKNNPYIYISGTFKNPKSTDKE
LPNQTIIRRYTYNKSTDTLEKPVDLLAGLPSSKDHQSGRLVIGPDQKIYYTIGDQ
GRNQLAYLFLPNQAQHTPTQQELNGKDYHTYMGKVLRLNLDGSIPKDNPSFNG
VVSHIYTLGHRNPQGLAFTPNGKLLQSEQGPNSDDEINLIVKGGNYGWPNVAG
YKDDSGYAYANYSAAANKTIKDLAQNGVKVAAGVPVTKESEWTGKNFVPPL
KTLYTVQDEDAPESGSPELGFSISGGVGGRGNPFRPDDDGIFVTRVQPEGPASKL
LQPGDKIIQANGYSFINIEHGQAVSLLKTFQNTVELIIVREVGNGAKQEIRVRVE
KDGGSGGVSSVPTNLEVVAATPTSLLISWDAYRELPVSYYRITYGETGGNSPVQ
EFTVPGSKSTATISGLKPGVDYTITVYAHYNYHYYSSPISINYRGSGPGYNYNDP
TCGEMTYICWPTVAPSSAYVYKGGKKAITGWENTLLVPSLKRGVIFRIKLDPTY
STTYDDAVPMFKSNNRYRDVIASPDGNVLYVLTDTAGNVQKDDGSVTNTLEN
PGSLIKFTYKAKHHEIHHH
ePDZ domain (SEQ ID NO: 26)
EDAPESPELGFSISGGVGGRGNPFRPDDDGIFVTRVQPEGPASKLLQPGDKIIQA
NGYSFINIEHGQAVSLLKTFQNTVELIIVREVGNGAKQEIRVRVEKDGGSGGVSS
VPTNLEVVAATPTSLLISWDAYRELPVSYYRITYGETGGNSPVQEFTVPGSKSTA
TISGLKPGVDYTITVYAHYNYHYYSSPISINYRGPG
PDZ peptide (ePDZ ligand, high affinity, strong peptide) (SEQ ID NO: 27)
RGSIDTWV
PDZ peptide (ePDZ ligand, Weak ligand, weak peptide) (SEQ ID NO: 28)
PQPVDSWV
ePDZ-GDH-CalM-Thrombin site-CalM peptide-ePDZ ligand (SEQ ID NO: 29)
DHHHHHHSPELGFSISGGVGGRGNPFRPDDDGIFVTRVQPEGPASKLLQPGDKII
QANGYSFINIEHGQAVSLLKTFQNTVELIIVREVGNGAKQEIRVRVEKDGGSGG
VSSVPTNLEVVAATPTSLLISWDAYRELPVSYYRITYGETGGNSPVQEFTVPGSK
STATISGLKPGVDYTITVYAHYNYHYYSSPISINYRGPDVPLIPSQFAKAKSENFD
KKVILSNLNKPHALLWGPDNQIWLTERATGKILRVNPESGSVKTVFQVPEIVND
ADGQNGLLGFAFHPDFKNNPYIYISGTFKNPKSTDKELPNQTIIRRYTYNKSTDT
LEKPVDLLAGLPSSKDHQSGRLVIGPDQKIYYTIGDQGRNQLAYLFLPNQAQHT
PTQQELNGKDYHTYMGKVLRLNLDGSIPKDNPSFNGVVSHIYTLGHRNPQGLA
FTPNGKLLQSEQGPNSDDEINLIVKGGNYGWPNVAGYKDDSGYAYANYSAAA
NKTIKDLAQNGVKVAAGVPVTKESEWTGKNFVPPLKTLYTVQDGSGGTEEQIA
EFKEAFSLFDKDGDGTITTKELGTVMRSLGQNPTEAELQDMINEVDADGNGTID
FPEFLTMMARKMKDTDSEEEIREAFRVFDKDGNGYISAAELRHVMTNLGEKLT
DEEVDEMIREADIDGDGQVNYEEFVQMMTAGGSGGYNYNDPTCGEMTYICWP
TVAPSSAYVYKGGKKAITGWENTLLVPSLKRGVIFRIKLDPTYSTTYDDAVPMF
KSNNRYRDVIASPDGNVLYVLTDTAGNVQKDDGSVTNTLENPGSLIKFTYKAK
LVPRGVKRRWKKNFIAVSAANRFKKISGGSGSGSGGSGTGSGSGSGGSTGGSGS
GGSRGSIDTWV
PQQ-GDH residues 1-329 (SEQ ID NO: 30)
DVPLIPSQFAKAKSENFDKKVILSNLNKPHALLWGPDNQIWLTERATGKILRVN
PESGSVKTVFQVPEIVNDADGQNGLLGFAFHPDFKNNPYIYISGTFKNPKSTDKE
LPNQTIIRRYTYNKSTDTLEKPVDLLAGLPSSKDHQSGRLVIGPDQKIYYTIGDQ
GRNQLAYLFLPNQAQHTPTQQELNGKDYHTYMGKVLRLNLDGSIPKDNPSFNG
VVSHIYTLGHRNPQGLAFTPNGKLLQSEQGPNSDDEINLIVKGGNYGWPNVAG
YKDDSGYAYANYSAAANKTIKDLAQNGVKVAAGVPVTKESEWTGKNFVPPL
KTLYTVQD
PQQ-GDH residues 331-454 (SEQ ID NO: 31)
YNYNDPTCGEMTYICWPTVAPSSAYVYKGGKKAITGWENTLLVPSLKRGVIFRI
KLDPTYSTTYDDAVPMFKSNNRYRDVIASPDGNVLYVLTDTAGNVQKDDGSV
TNTLENPGSLIKFTYKAK
TVMV cleavage site (SEQ ID NO: 32) ETVRFQS
Thrombin cleavage site (SEQ ID NO: 33) LVPRGV
Factor Xa cleavage sites IEGR (SEQ ID NO: 34) or IGDR (SEQ ID NO: 35)
Thrombin high affinity binding site (SEQ ID NO: 36) KTAPPFDFEAIPEEYL
Human IL18 dissociative sensor (SEQ ID NO: 37)
(GDH-ePDZ-Interleukin 18 binding protein)
DVPLIPSQFAKAKSENFDKKVILSNLNKPHALLWGPDNQIWLTERATGKILRVN
PESGSVKTVFQVPEIVNDADGQNGLLGFAFHPDFKNNPYIYISGTFKNPKSTDKE
LPNQTIIRRYTYNKSTDTLEKPVDLLAGLPSSKDHQSGRLVIGPDQKIYYTIGDQ
GRNQLAYLFLPNQAQHTPTQQELNGKDYHTYMGKVLRLNLDGSIPKDNPSFNG
VVSHIYTLGHRNPQGLAFTPNGKLLQSEQGPNSDDEINLIVKGGNYGWPNVAG
YKDDSGYAYANYSAAANKTIKDLAQNGVKVAAGVPVTKESEWTGKNFVPPL
KTLYTVQDEDAPESGSPELGFSISGGVGGRGNPFRPDDDGIFVTRVQPEGPASKL
LQPGDKIIQANGYSFINIEHGQAVSLLKTFQNTVELIIVREVGNGAKQEIRVRVE
KDGGSGGVSSVPTNLEVVAATPTSLLISWDAYRELPVSYYRITYGETGGNSPVQ
EFTVPGSKSTATISGLKPGVDYTITVYAHYNYHYYSSPISINYRGSGPGYNYNDP
TCGEMTYICWPTVAPSSAYVYKGGKKAITGWENTLLVPSLKRGVIFRIKLDPTY
STTYDDAVPMFKSNNRYRDVIASPDGNVLYVLTDTAGNVQKDDGSVTNTLEN
PGSLIKFTYKAKGSGGSGGSGSGGGAMVETKCPNLDIVTSSGEFHCSGCVEHMP
EFSYMYWLAKDMKSDEDTKFIEHLGDGINEDETVRTTDGGITTLRKVLHVTDT
NKFAHYRFTCVLTTLDGVSKKNIWLKKLAAALEHHHHHH
Interleukin-18-ePDZ peptide (SEQ ID NO: 38)
DGHHHHHHGSGGYFGKLESKLSVIRNLNDQVLFIDQGNRPLFEDMTDSDSRDN
APRTIFIISMYKDSQPRGMAVTISVKSEKISTLSSENKIISFKEMNPPDNIKDTKSDI
IFFQRSVPGHDNKMQFESSSYEGYFLASEKERDLFKLILKKEDELGDRSIMFTVQ
NEDGSGSGSGSGSGGRGSIDTWV
Auto-inhibited GDH-ePDZ peptide sensor-strong peptide (SEQ ID NO: 39)
(GDH-ePDZ-TVMV site-strong Peptide)
DVPLIPSQFAKAKSENFDKKVILSNLNKPHALLWGPDNQIWLTERATGKILRVN
PESGSVKTVFQVPEIVNDADGQNGLLGFAFHPDFKNNPYIYISGTFKNPKSTDKE
LPNQTIIRRYTYNKSTDTLEKPVDLLAGLPSSKDHQSGRLVIGPDQKIYYTIGDQ
GRNQLAYLFLPNQAQHTPTQQELNGKDYHTYMGKVLRLNLDGSIPKDNPSFNG
VVSHIYTLGHRNPQGLAFTPNGKLLQSEQGPNSDDEINLIVKGGNYGWPNVAG
YKDDSGYAYANYSAAANKTIKDLAQNGVKVAAGVPVTKESEWTGKNFVPPL
KTLYTVQDEDAPESGSPELGFSISGGVGGRGNPFRPDDDGIFVTRVQPEGPASKL
LQPGDKIIQANGYSFINIEHGQAVSLLKTFQNTVELIIVREVGNGAKQEIRVRVE
KDGGSGGVSSVPTNLEVVAATPTSLLISWDAYRELPVSYYRITYGETGGNSPVQ
EFTVPGSKSTATISGLKPGVDYTITVYAHYNYHYYSSPISINYRGSGPGYNYNDP
TCGEMTYICWPTVAPSSAYVYKGGKKAITGWENTLLVPSLKRGVIFRIKLDPTY
STTYDDAVPMFKSNNRYRDVIASPDGNVLYVLTDTAGNVQKDDGSVTNTLEN
PGSLIKFTYKAKGSGHHHHHHGSGETVRFQSSGSGGRGSIDTWV
Auto-inhibited GDH-ePDZ peptide sensor-weak peptide (SEQ ID NO: 40)
(GDH-ePDZ-TVMV site-weak Peptide)
DVPLIPSQFAKAKSENFDKKVILSNLNKPHALLWGPDNQIWLTERATGKILRVN
PESGSVKTVFQVPEIVNDADGQNGLLGFAFHPDFKNNPYIYISGTFKNPKSTDKE
LPNQTIIRRYTYNKSTDTLEKPVDLLAGLPSSKDHQSGRLVIGPDQKIYYTIGDQ
GRNQLAYLFLPNQAQHTPTQQELNGKDYHTYMGKVLRLNLDGSIPKDNPSFNG
VVSHIYTLGHRNPQGLAFTPNGKLLQSEQGPNSDDEINLIVKGGNYGWPNVAG
YKDDSGYAYANYSAAANKTIKDLAQNGVKVAAGVPVTKESEWTGKNFVPPL
KTLYTVQDEDAPESGSPELGFSISGGVGGRGNPFRPDDDGIFVTRVQPEGPASKL
LQPGDKIIQANGYSFINIEHGQAVSLLKTFQNTVELIIVREVGNGAKQEIRVRVE
KDGGSGGVSSVPTNLEVVAATPTSLLISWDAYRELPVSYYRITYGETGGNSPVQ
EFTVPGSKSTATISGLKPGVDYTITVYAHYNYHYYSSPISINYRGSGPGYNYNDP
TCGEMTYICWPTVAPSSAYVYKGGKKAITGWENTLLVPSLKRGVIFRIKLDPTY
STTYDDAVPMFKSNNRYRDVIASPDGNVLYVLTDTAGNVQKDDGSVTNTLEN
PGSLIKFTYKAKGSGHHHHHHGSGETVRFQSSGSGGPQPVDSWV
Interleukin-18 binding protein (SEQ ID NO: 41)
GAMVETKCPNLDIVTSSGEFHCSGCVEHMPEFSYMYWLAKDMKSDEDTKFIEH
LGDGINEDETVRTTDGGITTLRKVLHVTDTNKFAHYRFTCVLTTLDGVSKKNI
WLK
Interleukin-18 (SEQ ID NO: 42)
GYFGKLESKLSVIRNLNDQVLFIDQGNRPLFEDMTDSDSRDNAPRTIFIISMYKD
SQPRGMAVTISVKSEKISTLSSENKIISFKEMNPPDNIKDTKSDIIFFQRSVPGHDN
KMQFESSSYEGYFLASEKERDLFKLILKKEDELGDRSIMFTVQNEDG
GDH(1-153AA)-FRB (SEQ ID NO: 43)
DVPLIPSQFAKAKSENFDKKVILSNLNKPHALLWGPDNQIWLTERATGKILRVN
PESGSVKTVFQVPEIVNDADGQNGLLGFAFHPDFKNNPYIYISGTFKNPKSTDKE
LPNQTIIRRYTYNKSTDTLEKPVDLLAGLPSSKDHQSGRLVIGPGGSGSGSGGL
WHEMWHEGLEEASRLYFGERNVKGMFEVLEPLHAMMERGPQTLKETSFNQA
YGRDLMEAQEWCRKYMKSGNVKDLTQAWDLYYHVFRRISGKLAAALEHHH
HHH
GDH (1-153AA, Q76A, D143A,H144A)-TVMV cleavage site-FKBP-GDH (155-
454AA) (SEQ ID NO: 44)
DVPLIPSQFAKAKSENFDKKVILSNLNKPHALLWGPDNQIWLTERATGKILRVN
PESGSVKTVFQVPEIVNDADGANGLLGFAFHPDFKNNPYIYISGTFKNPKSTDKE
LPNQTIIRRYTYNKSTDTLEKPVDLLAGLPSSKAAQSGRLVIGPGGSGGETVRFQ
SGGSGSGGVQVETISPGDGRTFPKRGQTCWHYTGMLEDGKKFDSSRDRNKPFKF
MLGKQEVIRGWEEGVAQMSVGQRAKLTISPDVAYGATGHPGIIPPHATLVFDVELLK
LEGSGQKIYYTIGDQGRNQLAYLFLPNQAQHTPTQQELNGKDYHTYMGKVLRL
NLDGSIPKDNPSFNGVVSHIYTLGHRNPQGLAFTPNGKLLQSEQGPNSDDEINLI
VKGGNYGWPNVAGYKDDSGYAYANYSAAANKTIKDLAQNGVKVAAGVPVT
KESEWTGKNFVPPLKTLYTVQDTYNYNDPTCGEMTYICWPTVAPSSAYVYKG
GKKAITGWENTLLVPSLKRGVIFRIKLDPTYSTTYDDAVPMFKSNNRYRDVIAS
PDGNVLYVLTDTAGNVQKDDGSVTNTLENPGSLIKFTYKAKHHHHHH

### REFERENCES

(1) Turner, A. (2013) Biosensors: then and now. Trends Biotechnol. 31, 119-20.
(2) Ronkainen, N. J., Halsall, H. B., and Heineman, W. R. (2010) Electrochemical biosensors. Chem. Soc. Rev. 39, 1747-63.
(3) Yoo, E.-H., and Lee, S.-Y. (2010) Glucose biosensors: an overview of use in clinical practice. Sensors (Basel). 10, 4558-76.
(4) Hu, J., and Zhang, X.-E. (2011) Impact of epidemic rates of diabetes on the Chinese blood glucose testing market. J. Diabetes Sci. Technol. 5, 1294-9.
(5) Guo, Z., Johnston, W. A., Stein, V., Kalimuthu, P., Perez-Alcala, S., Bernhardt, P. V, and Alexandrov, K. (2016) Engineering PQQ-glucose dehydrogenase into an allosteric electrochemical Ca(2+) sensor. Chem. Commun. (Camb). 52, 485-8.
(6) Oubrie, A., Rozeboom, H. J., Kalk, K. H., Olsthoorn, A. J., Duine, J. A., and Dijkstra, B. W. (1999) Structure and mechanism of soluble quinoprotein glucose dehydrogenase. EMBO J. 18, 5187-94.
(7) Ishida, H., Nguyen, L. T., Gopal, R., Aizawa, T., and Vogel, H. J. (2016) Overexpression of Antimicrobial, Anticancer, and Transmembrane Peptides in Escherichia coli through a Calmodulin-Peptide Fusion System. J. Am. Chem. Soc. 138, 11318-26.
(8) Guo, Z., Murphy, L., Stein, V., Johnston, W. A., Alcala-Perez, S., and Alexandrov, K. (2016) Engineered PQQ-Glucose Dehydrogenase as a Universal Biosensor Platform. J. Am. Chem. Soc. 138, 10108-11.
(9) Huang, J., Koide, A., Makabe, K., and Koide, S. (2008) Design of protein function leaps by directed domain interface evolution. Proc. Natl. Acad. Sci. U.S.A 105, 6578-6583.
(10) Huang, J., Makabe, K., Biancalana, M., Koide, A., and Koide, S. (2009) Structural basis for exquisite specificity of affinity clamps, synthetic binding proteins generated through directed domain-interface evolution. J Mol Biol 392, 1221-1231.
(11) Huang, J., and Koide, S. (2010) Rational conversion of affinity reagents into label-free sensors for Peptide motifs by designed allostery. ACS Chem. Biol. 5, 273-277.
(12) Stein, V., and Alexandrov, K. (2014) Protease-based synthetic sensing and signal amplification. Proc. Natl. Acad. Sci. U.S.A 111, 15934-9.
(13) Nadler, D. C., Morgan, S.-A., Flamholz, A., Kortright, K. E., and Savage, D. F. (2016) Rapid construction of metabolite biosensors using domain-insertion profiling. Nat. Commun. 7, 12266.

1. An oxidoreductase enzyme comprising a heterologous amino acid sequence which is responsive to a peptide, wherein binding of the peptide to the heterologous amino acid sequence reversibly regulates catalytic activity of the enzyme.

2. The oxidoreductase enzyme of claim 1, which displays a reduction in catalytic activity in the absence of binding of said peptide to the heterologous amino acid sequence.

3. The oxidoreductase enzyme of claim 1 or 2, wherein the heterologous amino acid sequence is a calmodulin protein, or a functional fragment thereof.

4. The oxidoreductase enzyme of claim 3, which is responsive to a calmodulin-binding peptide, wherein binding of the peptide activates catalytic activity of the enzyme.

5. The oxidoreductase enzyme of claim 1, which displays a reduction in catalytic activity in the presence of binding of said peptide to the heterologous amino acid sequence.

6. The oxidoreductase enzyme of claim 5, wherein the heterologous amino acid sequence is an affinity clamp which binds said peptide.

7. The oxidoreductase enzyme of claim 5 or 6, which is responsive to a target molecule, wherein binding of the target molecule displaces said peptide to activate catalytic activity of the enzyme.

8. The oxidoreductase enzyme of any one of the preceding claims, comprising a binding moiety capable of interacting with a respective binding moiety on said peptide, wherein interaction between the binding moieties regulates catalytic activity of the enzyme.

9. The oxidoreductase enzyme of any one of claims 1 to 4, comprising a binding moiety capable of interacting with a respective binding moiety on said peptide, wherein catalytic activity of the enzyme is cooperatively enhanced by binding of the peptide and interaction between the binding moieties.

10. The oxidoreductase enzyme of claim 8 or 9, wherein said peptide is engineered to bind the heterologous amino acid sequence with an affinity insufficient to enhance catalytic activity in the absence of said interaction between binding moieties.

11. The oxidoreductase enzyme of any one of claims 8 to 10, wherein interaction of the binding moieties is dependent on presence of a target molecule, such that catalytic activity of the enzyme is enhanced in the presence of the target molecule.

12. The oxidoreductase enzyme of any one of the preceding claims, comprising one or more protease cleavage sites, wherein cleavage of a said site by a protease acts to regulate catalytic activity of the enzyme.

13. The oxidoreductase enzyme of any one of the preceding claims, wherein said peptide is covalently attached to, or forms part of a contiguous amino acid sequence of, said enzyme.

14. The oxidoreductase enzyme of claim 13, wherein said peptide is incapable of binding the heterologous amino acid sequence in the absence of a further molecule.

15. The oxidoreductase enzyme of claim 14, which comprises a moiety acting to prevent binding of said peptide to the heterologous amino acid sequence, wherein said moiety is displaced in the presence of the further molecule.

16. The oxidoreductase enzyme of claim 15, wherein said moiety is a binding moiety capable of interacting with a respective binding moiety on said further molecule, wherein interaction between the binding moieties releases said peptide to bind to the heterologous amino acid sequence.

17. The oxidoreductase enzyme of claim 15 or 16, wherein said moiety comprises one or more protease cleavage sites and said further molecule is a protease, wherein cleavage of a said site by said protease releases said peptide to bind to the heterologous amino acid sequence.

18. The oxidoreductase enzyme of any one of the preceding claims, which comprises said heterologous amino acid sequence at a location comprising one or more residues which influence substrate binding and/or catalytic activity of said enzyme.

19. The oxidoreductase enzyme of any one of the preceding claims, which is a glucose dehydrogenase (GDH) enzyme.

20. The GDH enzyme of claim 19, which comprises said heterologous amino acid sequence in a location corresponding to the loop connecting beta-sheets 4 and 5 of a PQQ-GDH.

21. An oxidoreductase enzyme comprising a heterologous amino acid sequence inserted at a location comprising one or more residues which influence substrate binding of said enzyme, wherein the heterologous amino acid sequence reversibly regulates the catalytic activity of the enzyme.

22. The oxidoreductase enzyme of claim 21, which is a glucose dehydrogenase (GDH) enzyme.

23. The oxidoreductase enzyme of claim 22, which comprises the heterologous amino acid sequence at a location corresponding to the loop connecting beta-sheets 4 and 5 of a PQQ-GDH.

24. A polypeptide comprising a first fragment sequence of an oxidoreductase enzyme, which is capable of non-covalently interacting with a polypeptide comprising a second fragment sequence of said enzyme to reconstitute a stable oxidoreductase enzyme, wherein the first and second fragment sequences represent sequences obtainable by cleavage of the enzyme at a location comprising one or more residues which influence substrate binding of said enzyme.

25. The polypeptide comprising a first fragment sequence of an oxidoreductase enzyme of claim 24, which is capable of reconstituting a stable catalytically active oxidoreductase enzyme with said polypeptide comprising a second fragment sequence of said enzyme.

26. The polypeptide comprising a first fragment sequence of an oxidoreductase enzyme of claim 25, which comprises one or more mutations which render the reconstituted stable GDH enzyme catalytically inactive.

27. The polypeptide comprising a first fragment sequence of an oxidoreductase enzyme of any one of claims 24-26, which comprises a binding moiety capable of interacting with a respective binding moiety comprised in said polypeptide comprising a second fragment of said enzyme, wherein said interaction between the binding moieties regulates catalytic activity of the reconstituted stable oxidoreductase enzyme.

28. The polypeptide comprising a first fragment sequence of an oxidoreductase enzyme of any one of claims 24-27, wherein said oxidoreductase enzyme is a GDH enzyme.

29. The polypeptide comprising a first fragment sequence of a GDH enzyme of claim 28, which represents a sequence obtainable by cleavage of the enzyme at a location corresponding to the loop connecting beta-sheets 4 and 5 of a PQQ-GDH.

30. A biosensor comprising an enzyme and a heterologous amino acid sequence that releasably maintains said enzyme in a catalytically inactive state in the presence of a peptide, wherein the heterologous amino acid sequence binds to the peptide to switch the enzyme from a catalytically active state to a catalytically inactive state.

31. A biosensor comprising an oxidoreductase enzyme of any one of claims 1 to 23 or the polypeptides comprising first and second fragment sequences as defined in any one of claims 24-29.

32. A composition or kit comprising the oxidoreductase enzyme of any one of claims 1 to 23, the polypeptides comprising first and second fragment sequences as defined in any one of claims 24-29, the biosensor comprising an enzyme of claim 30, or the biosensor of claim 31.

33. The composition or kit of claim 32 comprising said oxidoreductase enzyme, biosensor comprising an enzyme, or biosensor, further comprising a said peptide acting to regulate catalytic activity of said enzyme by binding to said heterologous amino acid sequence.

34. The composition or kit of claim 33, wherein said oxidoreductase enzyme or said biosensor comprising an enzyme comprises a binding moiety and said peptide comprises a respective binding moiety, wherein interaction between the binding moieties regulates catalytic activity of the enzyme.

35. The composition or kit of any one of claims 32 to 34, wherein said oxidoreductase enzyme, biosensor comprising an enzyme, biosensor, or said polypeptide comprising a first or second fragment sequence comprises one or more protease cleavage sites, wherein cleavage of a said site by a protease acts to regulate catalytic activity of the enzyme, and said composition or kit further comprises a said protease capable of cleaving said site.

36. The composition or kit of claim 35, wherein said protease comprises a binding moiety and said oxidoreductase enzyme, biosensor comprising an enzyme, biosensor, or said polypeptide comprising a first or second fragment sequence comprises a respective binding moiety, and/or said protease comprises an inhibitory moiety acting to prevent cleavage activity of said protease, wherein said inhibitory moiety is capable of being displaced in the presence of said enzyme, such that the protease is able to cleave said site.

37. The composition or kit of any one of claims 32-36, further comprising a substrate molecule for said enzyme.

38. A method of detecting a target molecule, comprising contacting the oxidoreductase enzyme of any one of claims 1 to 23, the polypeptides comprising first and second fragment sequences as defined in any one of claims 24-29, the biosensor comprising an enzyme of claim 30, or the biosensor of claim 31 with a sample under conditions suitable for detection of the presence or absence of the target molecule in the sample.

39. A method of diagnosis of a disease or condition in an organism, comprising contacting the oxidoreductase enzyme of any one of claims 1 to 23, the polypeptides comprising first and second fragment sequences as defined in any one of claims 24-29, the biosensor comprising an enzyme of claim 30, or the biosensor of claim 31 with a sample obtained from the organism under conditions suitable for detection of the presence or absence of the target molecule in the sample, wherein presence or absence of the target molecule in the sample is indicative of whether the organism has, or is at risk of having, said disease or condition.

40. A detection device that comprises a cell or chamber that comprises the oxidoreductase enzyme of any one of claims 1 to 23, the polypeptides comprising first and second fragment sequences as defined in any one of claims 24-29, the biosensor comprising an enzyme of claim 30, or the biosensor of claim 31.

41. A nucleic acid encoding the oxidoreductase enzyme of any one of claims 1 to 23, a polypeptide comprising a first or second fragment sequence as defined in any one of claims 24-29, the biosensor comprising an enzyme of claim 30, or the biosensor of claim 31.


SEQUENCE LISTING (44 sequences)

SEQ ID NO: 1 - protein, 454 aa, Artificial Sequence
  mature PQQ-GDH protein
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAK

SEQ ID NO: 2 - protein, 478 aa, Artificial Sequence
  Protein sequence before cleavage of signal sequence
MNKHLLAKIALLGAAQLVTLSAFADVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAK

SEQ ID NO: 3 - protein, 143 aa, Artificial Sequence
  Calmodulin protein
TEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTA

SEQ ID NO: 4 - protein, 26 aa, Artificial Sequence
  CaM-binding peptide from structure PDB2BBM
KRRWKKNFIAVSAANRFKKISSSGAL

SEQ ID NO: 5 - protein, 21 aa, Artificial Sequence
  Modified CaM-BP
KRRWKKNFIAVSAANRFKKIS

SEQ ID NO: 6 - protein, 16 aa, Artificial Sequence
  Modified CaM-BP
KRRWKKNFIAVSAANR

SEQ ID NO: 7 - protein, 10 aa, Artificial Sequence
  Modified CaM-BP
KRRWKKNFIA

SEQ ID NO: 8 - protein, 16 aa, Artificial Sequence
  Preferred Modified CaM-BP
KRRWKKNFIAVASASA

SEQ ID NO: 9 - protein, 611 aa, Artificial Sequence
  GDH-CaM (first generation)
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDGSGSGGSGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKHHHHHH

SEQ ID NO: 10 - protein, 611 aa, Artificial Sequence
  GDH-CaM (second generation)
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSGGYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKHHHHHH

SEQ ID NO: 11 - protein, 730 aa, Artificial Sequence
  GDH-CaM-FKBP12
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSGGYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKGGSGGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEKLAAALEHHHHHH

SEQ ID NO: 12 - protein, 137 aa, Artificial Sequence
  FRB-CaM-BP
AHHHHHHSSGTRVAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGSGSGSGGSGGKRRWKKNFIAVASASA

SEQ ID NO: 13 - protein, 107 aa, Artificial Sequence
  FKPB12
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 14 - protein, 91 aa, Artificial Sequence
  FRB
LWHEMWHEGLEEASRLYFGERNVKGMFEVLEPLHAMMERGPQTLKETSFNQAYGRDLMEAQEWCRKYMKSGNVKDLTQAWDLYYHVFRRIS

SEQ ID NO: 15 - protein, 509 aa, Artificial Sequence
  SUMO-CN alpha subunit
MGSSHHHHHHGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGTSEPKAIDPKLSTTDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDVYNNKAAVLKYENNVMNIRQFNCSPHPYWLPNFMDVFTWSLPFVGEKVTEMLVNVLNICSDDELGSEEDGFDGATAAARLVTAGLVLA

SEQ ID NO: 16 - protein, 209 aa, Artificial Sequence
  CN beta subunit-CalM peptide
DGHHHHHHGGNEASYPLEMCSHFDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNLKDTQLQQIVDKTIINADKDGDGRISFEEFCAVVGGLDIHKKMVVDVGGSGGSGSGSGGSGGKRRWKKNFIAVASASA

SEQ ID NO: 17 - protein, 108 aa, Artificial Sequence
  SUMO
MGSSHHHHHHGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 18 - protein, 401 aa, Artificial Sequence
  Calcineurin alpha
TSEPKAIDPKLSTTDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDVYNNKAAVLKYENNVMNIRQFNCSPHPYWLPNFMDVFTWSLPFVGEKVTEMLVNVLNICSDDELGSEEDGFDGATAAARLVTAGLVLA

SEQ ID NO: 19 - protein, 168 aa, Artificial Sequence
  Calcineurin beta
NEASYPLEMCSHFDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNLKDTQLQQIVDKTIINADKDGDGRISFEEFCAVVGGLDIHKKMVVDV

SEQ ID NO: 20 - protein, 745 aa, Artificial Sequence
  GDH-CaM-VHH1
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSGGYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKGSGSGGQVQLVESGGGTVPAGGSLRLSCAASGNTLCTYDMSWYRRAPGKGRDFVSGIDNDGTTTYVDSVAGRFTISQGNAKNTAYLQMDSLKPDDTAMYYCKPSLRYGLPGCPIIPWGQGTQVTVSSKLAAALEHHHHHH

SEQ ID NO: 21 - protein, 116 aa, Artificial Sequence
  VHH2-CaM-BP
DGHHHHHHGSGDTTVSEPAPSCVTLYQSWRYSQADNGCAETVTVKVVYEDDTEGLCYAVAPGQITTVGDGYIGSHGHARYLARCLGGSGGSGSGSGGSGGKRRWKKNFIAVASASA

SEQ ID NO: 22 - protein, 121 aa, Artificial Sequence
  VHH1
QVQLVESGGGTVPAGGSLRLSCAASGNTLCTYDMSWYRRAPGKGRDFVSGIDNDGTTTYVDSVAGRFTISQGNAKNTAYLQMDSLKPDDTAMYYCKPSLRYGLPGCPIIPWGQGTQVTVSS

SEQ ID NO: 23 - protein, 74 aa, Artificial Sequence
  VHH2
DTTVSEPAPSCVTLYQSWRYSQADNGCAETVTVKVVYEDDTEGLCYAVAPGQITTVGDGYIGSHGHARYLARCL

SEQ ID NO: 24 - protein, 660 aa, Artificial Sequence
  GDH-ePDZ peptide sensor first version
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDEDAPESPELGFSISGGVGGRGNPFRPDDDGIFVTRVQPEGPASKLLQPGDKIIQANGYSFINIEHGQAVSLLKTFQNTVELIIVREVGNGAKQEIRVRVEKDGGSGGVSSVPTNLEVVAATPTSLLISWDAYRELPVSYYRITYGETGGNSPVQEFTVPGSKSTATISGLKPGVDYTITVYAHYNYHYYSSPISINYRGPGYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKHHHHHH

SEQ ID NO: 25 - protein, 664 aa, Artificial Sequence
  GDH-ePDZ peptide sensor second version
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDEDAPESGSPELGFSISGGVGGRGNPFRPDDDGIFVTRVQPEGPASKLLQPGDKIIQANGYSFINIEHGQAVSLLKTFQNTVELIIVREVGNGAKQEIRVRVEKDGGSGGVSSVPTNLEVVAATPTSLLISWDAYRELPVSYYRITYGETGGNSPVQEFTVPGSKSTATISGLKPGVDYTITVYAHYNYHYYSSPISINYRGSGPGYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKHHHHHH

SEQ ID NO: 26 - protein, 201 aa, Artificial Sequence
  ePDZ domain
EDAPESPELGFSISGGVGGRGNPFRPDDDGIFVTRVQPEGPASKLLQPGDKIIQANGYSFINIEHGQAVSLLKTFQNTVELIIVREVGNGAKQEIRVRVEKDGGSGGVSSVPTNLEVVAATPTSLLISWDAYRELPVSYYRITYGETGGNSPVQEFTVPGSKSTATISGLKPGVDYTITVYAHYNYHYYSSPISINYRGPG

SEQ ID NO: 27 - protein, 8 aa, Artificial Sequence
  PDZ peptide (ePDZ ligand, high affinity)
RGSIDTWV

SEQ ID NO: 28 - protein, 8 aa, Artificial Sequence
  PDZ peptide (ePDZ ligand, Weak ligand)
PQPVDSWV

SEQ ID NO: 29 - protein, 872 aa, Artificial Sequence
  ePDZ-GDH-CalM-Thrombin site-CalM peptide-ePDZ ligand
DHHHHHHSPELGFSISGGVGGRGNPFRPDDDGIFVTRVQPEGPASKLLQPGDKIIQANGYSFINIEHGQAVSLLKTFQNTVELIIVREVGNGAKQEIRVRVEKDGGSGGVSSVPTNLEVVAATPTSLLISWDAYRELPVSYYRITYGETGGNSPVQEFTVPGSKSTATISGLKPGVDYTITVYAHYNYHYYSSPISINYRGPDVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSGGYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKLVPRGVKRRWKKNFIAVSAANRFKKISGGSGSGSGGSGTGSGSGSGGSTGGSGSGGSRGSIDTWV

SEQ ID NO: 30 - protein, 329 aa, Artificial Sequence
  PQQ-GDH residues 1-329
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQD

SEQ ID NO: 31 - protein, 124 aa, Artificial Sequence
  PQQ-GDH residues 331-454
YNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAK

SEQ ID NO: 32 - protein, 7 aa, Artificial Sequence
  TVMV cleavage site
ETVRFQS

SEQ ID NO: 33 - protein, 6 aa, Artificial Sequence
  Thrombin cleavage site
LVPRGV

SEQ ID NO: 34 - protein, 4 aa, Artificial Sequence
  Factor Xa cleavage site (1)
IEGR

SEQ ID NO: 35 - protein, 4 aa, Artificial Sequence
  Factor Xa cleavage site (2)
IGDR

SEQ ID NO: 36 - protein, 16 aa, Artificial Sequence
  Thrombin high affinity binding site
KTAPPFDFEAIPEEYL

SEQ ID NO: 37 - protein, 792 aa, Artificial Sequence
  Human IL18 dissociative sensor
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDEDAPESGSPELGFSISGGVGGRGNPFRPDDDGIFVTRVQPEGPASKLLQPGDKIIQANGYSFINIEHGQAVSLLKTFQNTVELIIVREVGNGAKQEIRVRVEKDGGSGGVSSVPTNLEVVAATPTSLLISWDAYRELPVSYYRITYGETGGNSPVQEFTVPGSKSTATISGLKPGVDYTITVYAHYNYHYYSSPISINYRGSGPGYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKGSGGSGGSGSGGGAMVETKCPNLDIVTSSGEFHCSGCVEHMPEFSYMYWLAKDMKSDEDTKFIEHLGDGINEDETVRTTDGGITTLRKVLHVTDTNKFAHYRFTCVLTTLDGVSKKNIWLKKLAAALEHHHHHH

SEQ ID NO: 38 - protein, 189 aa, Artificial Sequence
  Interleukin-18-ePDZ peptide
DGHHHHHHGSGGYFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNEDGSGSGSGSGSGGRGSIDTWV

SEQ ID NO: 39 - protein, 690 aa, Artificial Sequence
  Auto-inhibited GDH-ePDZ peptide sensor-strong peptide
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDEDAPESGSPELGFSISGGVGGRGNPFRPDDDGIFVTRVQPEGPASKLLQPGDKIIQANGYSFINIEHGQAVSLLKTFQNTVELIIVREVGNGAKQEIRVRVEKDGGSGGVSSVPTNLEVVAATPTSLLISWDAYRELPVSYYRITYGETGGNSPVQEFTVPGSKSTATISGLKPGVDYTITVYAHYNYHYYSSPISINYRGSGPGYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKGSGHHHHHHGSGETVRFQSSGSGGRGSIDTWV

SEQ ID NO: 40 - protein, 690 aa, Artificial Sequence
  Auto-inhibited GDH-ePDZ peptide sensor-weak peptide
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDEDAPESGSPELGFSISGGVGGRGNPFRPDDDGIFVTRVQPEGPASKLLQPGDKIIQANGYSFINIEHGQAVSLLKTFQNTVELIIVREVGNGAKQEIRVRVEKDGGSGGVSSVPTNLEVVAATPTSLLISWDAYRELPVSYYRITYGETGGNSPVQEFTVPGSKSTATISGLKPGVDYTITVYAHYNYHYYSSPISINYRGSGPGYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKGSGHHHHHHGSGETVRFQSSGSGGPQPVDSWV

SEQ ID NO: 41 - protein, 109 aa, Artificial Sequence
  Interleukin-18 binding protein
GAMVETKCPNLDIVTSSGEFHCSGCVEHMPEFSYMYWLAKDMKSDEDTKFIEHLGDGINEDETVRTTDGGITTLRKVLHVTDTNKFAHYRFTCVLTTLDGVSKKNIWLK

SEQ ID NO: 42 - protein, 159 aa, Artificial Sequence
  Interleukin-18
GYFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNEDG

SEQ ID NO: 43 - protein, 267 aa, Artificial Sequence
  GDH(1-153AA)-FRB
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPGGSGSGSGGLWHEMWHEGLEEASRLYFGERNVKGMFEVLEPLHAMMERGPQTLKETSFNQAYGRDLMEAQEWCRKYMKSGNVKDLTQAWDLYYHVFRRISGKLAAALEHHHHHH

SEQ ID NO: 44 - protein, 587 aa, Artificial Sequence
  GDH(1-153AA, Q76A, D143A,H144A)-TVMV cleavage site-FKBP-GDH(155-454AA)
DVPLIPSQFAKAKSENFDKKVILSNLNKPHALLWGPDNQIWLTERATGKILRVNPESGSVKTVFQVPEIVNDADGANGLLGFAFHPDFKNNPYIYISGTFKNPKSTDKELPNQTIIRRYTYNKSTDTLEKPVDLLAGLPSSKAAQSGRLVIGPGGSGGETVRFQSGGSGSGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGSGQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKHHHHHH